(19) United States
(12) Patent Application Publication
(10) Pub. No.: US 2003/0023386 A1
(43) Pub. Date: Jan. 30, 2003

(54) Metabolome profiling methods using chromatographic and spectroscopic data in pattern recognition analysis

(76) Inventors: Nelly Aranibar, Lawrenceville, NJ (US); Karl-Heinz Ott, Lawrenceville, NJ (US); Gerald Stockton, Yardley, PA (US)

Correspondence Address: William L. Warren, Esq., SUTHERLAND ASBILL & BRENNAN LLP, 999 Peachtree Street, NE, Atlanta, GA, 30309-3996, US

(21) Appl. No.: 10/051,615

(22) Filed: Jan. 18, 2002

Related U.S. Application Data
(60) Provisional application No. 60/262,531, filed on Jan. 18, 2001.

(51) Int. Cl. G06F 19/00; G01N 33/48; G01N 33/50

(52) U.S. Cl. 702/19

(57) ABSTRACT

Methods are provided that apply neural network technology to recognize small metabolic changes in microorganisms, plants or animals to detect changes induced by pesticide (herbicide, insecticide, fungicide) treatment, genetic modification, environmental stress, and other external or internal factors that have influence on metabolite concentrations. The method implements recognition of nuclear magnetic resonance spectra, mass spectra, and/or chromatograms of crude plant extracts and association of such spectra or chromatograms with the treatment of tissue before harvest. The spectra and chromatograms have information of all the metabolites above a concentration threshold contained in the plant tissue extract. The method applies mathematical models to the very complex plant tissue extract and allows the detection of treatments with bioregulators such as pesticides, or genetic modifications such as gene insertions or deletions.

### CROSS-REFERENCE TO RELATED APPLICATIONS

This application claims priority to U.S. Provisional Application No. 60/262,531 filed Jan. 18, 2001.

### FIELD OF THE INVENTION

The present invention applies to spectroscopic and/or chromatographic techniques used in combination with neural network technology to recognize small metabolic changes in a sample of an organism, and to detect and classify changes induced by treatment of said organism, gene alteration, genetic modification, stress, and other external or internal forces that have influence on the concentrations of the pool of metabolites in the organism.

### BACKGROUND ART

Over the years, many spectroscopic techniques have been used to diagnose specific diseases or detect abnormal samples in a population of a group of samples, tissues, microbes, polymers, etc. Often Neural Networks (NN), Principal Components Analysis (PCA) and similar techniques have been shown to provide useful means for classifying such spectral information. Nuclear Magnetic Resonance (NMR) combined with pattern recognition has most widely been used in assaying of human diseases such as brain cancer where automated analysis of (NMR) spectra has been shown to allow distinction between normal and diseased tissue.

NMR combined with Pattern Recognition has also been used for the analysis and prediction of mammalian toxicity by utilizing urine samples from treated and untreated animals. Specific metabolites will show up in the samples indicating active detoxification in the liver. Individual identification and quantification of such metabolites is usually attempted. Those approaches are all intended to provide diagnostic tools comparing/distinguishing normal and disease states.

There are a few examples where a generalized classification scheme has been attempted as utilized in the present invention. The scope and implementation of the approaches mentioned above, however, differ largely from the scope of the present invention. Previously reported approaches, while similar in the underlying techniques used, i.e., use of NMR and Artificial Neural Network (ANN), have focussed on identification of specific toxicological parameters like target organ specificity from analysis of specific toxin metabolites. The present invention classifies biochemical pathway activity by monitoring the overall composition of the natural metabolite levels. Furthermore, sample and data analysis requirements are largely divergent between the present approach, i.e. tissue samples or extracts of tissue samples versus body fluids (urine). As used herein, the term “metabolite profiling” refers to those methods reported in the literature that focus on identification and/or quantification of specific reporting metabolites. The method described in the present invention that analyses the composition profile of all metabolites will be referred to as “metabolic profiling”. This reflects the difference between the prior art approaches to detect a set of metabolites as a diagnostic tool versus the present approach of using the profile of all metabolites to classify metabolic states. It should be noted that some of the literature does not differentiate these terms in a strict sense and many methods that are tailored to detect a set of metabolites are still referred to as metabolic profiling methods.

Plant References

Since earlier methods are usually targeted to mammalian systems, there are no examples of attempts to use ‘metabolic’ profiling to classify genetically altered organisms. One particular reference relating to plants is U.S. Pat. No. 5,900,634 for a device encompassing spectroscopy and a neural net for the analysis of food, fertilizers and pharmaceuticals. Other patents describe various combinations of analytical techniques and chemometric analysis or neutral networks to identify organisms, their origins, or food quality/contamination.

There are two relevant papers from the journal literature. J. Lozano, et. al., published a paper in 1995 on modeling metabolic energy of barley using twelve parameters. H. Sauter's paper entitled “Metabolic profiling of Plants: A new diagnostic technique” uses GC-MS and a computer for metabolite profiling of herbicide-treated barley seedlings. These journal references on plant applications involve the use of an analytical technique to measure a specific compound or related set of compounds. A recent publication by S. J. W. Hole et al. describes the use of NMR spectroscopy combined with PCA, PLS (partial least squares), or SIMCA (soft independent modeling of class analogy), which are multivariate statistical and clustering methods, to investigate herbicide mode of action in plants. However, such methods become increasingly impractical when more than a few MOAs are simultaneously tested. In general, in the scientific literature, the information is used to identify and classify plants, to predict the toxicity of chemicals (structure-activity relationships), to determine food quality (origin of product, adulteration, and contamination), and to analyze environmental pollutants.

Mammalian/Microbial/Pharmaceutical References

There are a number of relevant patents in the pharmaceutical area: M. J. Ala-Korpela describes the use of NMR and a Neural Networks to classify and quantify human brain metabolism (U.S. Pat. No. 5,887,588); H. K. Beving describes a system for a diagnostic process for cells and tissues (U.S. Pat. No. 5,687,716); Cedars-Sinai Medical Center describes a monitor and method for determining the metabolic state of an organ based on the fluorescence of NADH (U.S. Pat. No. 5,456,252); ESA Inc. uses pattern recognition from liquid chromatography with electrochemical detection to identify metabolites for use as a diagnostic technique. Nicholson's group has used some NMR/ANN based classification methods to studying toxin-induced changes in urine samples for diagnostic purposes.

There is some non-patent literature on the use of neural networks for metabolic/metabolite profiling in mammalian (human) [Ala-Korpela, 1997; Bakken, 1999; Bamforth, 1999; El-Deredy, 1997; Kaartinen, 1998] and some microbial (fermentation) organisms [Hagimori, 1993]. It is generally for specific organs and useful in the areas of diagnosis, pharmacokinetics and pharmarcodynamics [Gobburu, 1996; and metabolic models [Mendes, 1996].

Genetic alterations and some pesticide treatments will introduce only small changes in the metabolic profile. Such small changes must be isolated from a variety of other factors such as environmental conditions, which remain unchanged. The ability to grow plants and microorganism under controlled conditions distinguishes this approach from applications in toxicology and human disease where conditions may vary widely. The present approach thereby encompasses a much more detailed and sensitive analysis with many more categories than a diagnostic tool which, for example, is specifically designed to recognize the existence or non-existence of a brain tumor. The present approach utilizes the wealth of information that is present in the sum of all metabolites and their ratios to one another while eliminating the need for elaborate separation steps and individual identification of one or more reporter compounds.

The present approach is also novel as it encompasses a screening method to recognize an almost unlimited variety of treatments and environmental factors, gene and genetic modifications and alterations. The present approach also has the potential to be applied as a high-throughput screen since all steps can be automated if necessary. The approach described herein is preferably limited to organisms that can be grown and sampled under controlled conditions. This differentiates the present method further from applications in human diagnosis and toxicology studies.

Artificial Neural Networks

Artificial neural networks (ANN) have historically been greatly motivated by the attempt to model the high performance of the human brain in highly complex cognitive tasks like visual and auditory pattern recognition. However, most current ANN architectures do not try to closely imitate their biological model but rather can be regarded simply as a class of parallel algorithms.

In these models, knowledge is usually distributed throughout the net and is stored in the structure of the topology and the weights of the links. The networks are organized by (automated) training methods, which greatly simplify the development of specific applications. Vague conclusions and associative recall, i.e. exact match vs. best match, replace classical logic in ordinary Artificial Intelligence (AI) systems. This is a big advantage in all situations where no clear set of logical rules can be given. The inherent fault tolerance of connectionist models is another advantage. Furthermore, neural nets can be made tolerant against noise in the input, e.g. usually only the quality of the output degrades with increased noise. Their vagueness and associative nature make ANNs most suitable for the task to associate a similar spectrum of an organism or a crude extract of an organism, with a reference. The inherent variability between individual organisms, variations between batches and experimental noise require such a fault tolerant method.

Neural Network Terminology

Neural networks comprise of a variety of related techniques that are described in many monographs. One of the most comprehensive, and very recent monographs that explains the various techniques and components very well is A. Zell, Simulation Neuronaler Netze, R. Oldenbourg Verlag, Muenchen, Wien.

A typical NN consists of units and directed, weighted links (connections) between them. In analogy to activation passing in biological neurons, each unit receives a net input that is computed from the weighted outputs of prior units with connections leading to this unit. See FIG. 1. A Small Neural Network with Three Layers of Units.

The actual information processing within the units is modeled using both the activation function and the output function. The activation function first computes the net input of the unit from the weighted output values of prior units, then computes the new activation from this net input (and possibly its previous activation). The output function takes this result to generate the output of the unit.

Three types of units are distinguished based on their function within the net:

Units whose activation are the problem input for the net are called input units;

Units whose output represent the output of the net output units;

Units between input and output units, which are not visible from the outside, called hidden units.

There are connections between units of different layers. The direction of a connection shows the direction of the transfer of activation. Connections, called recursive connections, with identical source and target are possible. Each connection has a weight (or strength) value assigned to it. The effect of the output of one unit on the successor unit is defined by this value. If the value is negative, and then the connection has an inhibitory effect, i.e. the connection decreases the activity of the target unit. If the value is positive, then the connection has an excitatory or activity enhancing effect. The most frequently used network architecture is built hierarchically bottom-up. The input into a unit comes only from the units of preceding layers. These networks are also called feed-forward nets because of the unidirectional flow of information within the net. In many models a full connectivity between all units of adjoining levels is assumed but it can be advantageous to “prune” weak connections to improve performance if many units are in use.

Pattern Recognition Approaches

In the 1999 review “Metabonomics: understanding the metabolic responses of living systems to pathophysiological stimuli via multivariate statistical analysis of biological NMR spectroscopic data”, [Xenobiotics, 1999, 29, p.1181] Nicholson et al. “proposed a new NMR-based ‘metabonomic’ approach that is aimed at the augmentation and complementation of the information provided by measuring the genetic and proteomic responses to xenobiotic exposure.” He defines Metabonomics as “the quantitative measurement of the dynamic multiparametric metabolic response of living systems to pathophysiological stimuli or genetic modification.” He identifies metabonomics, as many authors before him, as “ . . . identifying, quantifying, and cataloging the history of the time-related metabolic changes . . . ” and proposes to apply NMR and multivariate statistical models, in particular Principal Component Analysis (PCA), to assay toxicity of drugs in a rat model. Nicholson et al. wrote that they foresee “the number of applications to increase in parallel with ongoing developments in instrumentation and techniques. In particular, the development of computer-based Pattern Recognition and expert systems for data analysis is expected to make major contributions to the advancements of NMR-based metabolic science. Other important areas accessible to metabonomic investigation include studies on biochemical consequences of genetic modification . . . ”.

The method described in the present invention enables an approach to study biochemical consequences in non-mammalian systems, and also to further build a generalized high-throughput assay system for many different genes and pesticides in non-mammalian organisms.

In particular, the method described here does not assume any prior knowledge about the nature and function of the test gene or pesticide. In contrast to the approach outlined by Nicholson et al., the method disclosed herein does not specifically rely on the quantification of many parameters but qualitatively recognizes the history of metabolic events based on a generalized classification scheme.

### SUMMARY

The present invention describes a metabolic profiling method for recognizing the metabolic state of biological, plant or microbial samples using spectroscopic and/or chromatographic methods and pattern recognition techniques.

The present invention encompasses a metabolic profiling method for recognizing and classifying environmental factors (e.g. stress, compound treatment) occurring during the development of an organism by using spectroscopic and/or chromatographic methods and pattern recognition techniques on samples of these organisms.

The present invention also includes the application of the metabolic profiling method for identification of gene alterations, genetic alterations or modifications, or identification and classification of variations in genotype, phenotype, developmental stage, or other factors that are reflected in the metabolic composition of the organism.

The invention also describes a metabolic response database developed from bioregulator treatments, specific gene modifications, gene level alterations and/or interruptions in metabolic pathways that induce positive/negative response in spectral components. It is within the scope of the invention to apply those techniques alone or in combination to plants, fungi, insects, and microorganisms.

The present invention describes and trains a NN designed to detect metabolic changes in microorganisms and/or plants from the metabolic response database, which correlates spectral response with a cellular state or treatment.

Also, we introduce a novel generalized, high-throughput method and/or assay system for determining the mode-of-action of a compound from analysis of the metabolic changes, spectral correlation and interruptions identified in the metabolic response database or by applying pattern recognition methods to cluster metabolic profiles.

The method described here is not limited to identifying specific metabolites, as in the toxicology studies, nor does it relate to a specific phenotype, as in the disease diagnosis.

The present invention describes a method for determining the influence of environmental stress factors in plants/microorganisms as deduced from their metabolic response.

Additionally, the invention describes a method to compare the profile of protein expression with the protein product in genetically modified plants/microorganisms.

### BRIEF DESCRIPTION OF THE DRAWINGS

FIG. 1. A Small Network with Three Layers of Units

FIG. 2. Proton NMR spectra of corn extracts. The plants had been treated with different herbicides, as indicated in each spectrum label. The central water peak has been removed from the spectrum for scaling and processing.

FIG. 3a., 3b. Designed-to-Fail Example of a network training/validation run. In the first part the spectra are listed that have been used to train a NN. PURSUIT® herbicide (imazethapyr) treated samples of batch na030100 have been recorded at a 3 K higher temperature. As shown in the lower part of the table, the NN fails to classify spectra of PURSUIT® treated samples recorded at a lower temperature (na022400). However, all other datasets are correctly recognized.

FIG. 4a., 4b. Blind Test of Four Different Compounds with AHAS Inhibition Mode-of-Action.

FIG. 5. Raw “Confusion Matrix” from Calculation A (Number of Plants in Class).

FIG. 6. Raw “Confusion Matrix” from Calculation B (Number of Plants in Class).

FIG. 7. Confusion Matrix From Calculation A.

FIG. 8. Confusion Matrix From Calculation B (Percentage of Plants in Class).

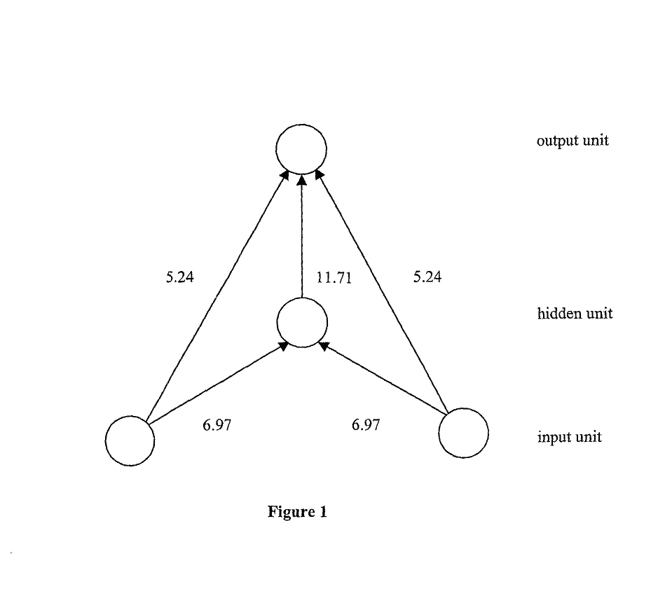

### DETAILED DESCRIPTION OF THE INVENTION

The present invention describes a method that is different in its focus, scope and implementation to published and patented methods. The method described herein specifically encompasses identification of genetic modifications using metabolic profiling or metabolite profiling techniques. It is within the scope of the invention to apply those techniques alone or in combination to plants, fungi, insects, and microorganisms to detect and classify compounds and/or genetic modifications for their activity, function and mode-of-action.

We introduce a novel, generalized, high-throughput method that uses information generated from changes in the overall profile of metabolite pool distributions. These changes are caused by the interrelated changes in activities of many pathways rather than changes in individually traced metabolites. The information can be used not only to classify bioregulators but also to classify genetic modification in terms of their ability to affect certain interconnected pathways. The classification is according to the changes in the natural metabolic composition due to direct and indirect changes in pathway activity and the resulting alteration in the composition of many different, unclassified metabolites. The method described here is neither limited to identifying specific metabolites as in the toxicology studies nor does it relate to a specific phenotype as in the disease diagnosis. Also, the method not only identifies the treatment in the sense of a specific diagnostic tool for a predefined phenotype/pathological state, but also allows screening for unspecified changes upon treatment with unknown compounds or genetic modification.

The present invention provides a metabolic profiling method for identifying a metabolic state of a subject biological sample. The metabolic state may also be termed the “metabalome” of the sample or organism. The metabolic state of the subject biological sample may be spontaneous (e.g., due to natural or introduced genetic alterations) or induced by an extraneous compound such as a bioregulator (e.g., a herbicide, growth factor, transcription factor, etc.) or other environmental stimuli (e.g., temperature, moisture, salinity, etc.).

The method comprises analyzing in an automated pattern recognition system, such as a neural network described herein, data obtained from the subject biological sample by a spectroscopic or chromatographic technique in comparison to data obtained from a plurality of other known biological samples by the spectroscopic or chromatographic technique to determine a comparable metabolic state. The data obtained is a compilation of a plurality of observed metabolites.

In this method, the biological samples are obtained from organisms grown under controlled conditions, as described further in the examples herein. Controlled conditions refers to the environment of the organisms being substantially identical in order to minimize extraneous metabolic differences due to non-subject parameters.

Furthermore, in certain embodiments, the chromatographic technique for obtaining data is gas chromatography. In certain embodiments, the spectroscopic technique is nuclear magnetic resonance spectroscopy or mass spectroscopy. In other embodiments, the technique for obtaining data is some combination of any chromatographic or spectroscopic technique.

The invention provides that metabolic profile can result from a metabolic state selected from the group consisting of: a. inhibition of acetyl CoA carboxylase (ACCase); b. inhibition of acetolactate synthase (ALS) or acetohydroxyacid synthase (AHAS); c. inhibition of photosynthesis at photosystem II; d. photosystem-I-electron diversion; e. inhibition of protoporphyrinogen oxidase (PPO); f. inhibition of carotenoid biosynthesis at the phytoene desaturase step (PDS); g. inhibition of 4-hydroxyphenyl-pyruvate-dioxygenase (4-HPPD); h. inhibition of carotenoid biosynthesis; i. inhibition of EPSP synthase; j. inhibition of glutamine synthetase; k. inhibition of DHP (dihydropteroate) synthase; l. microtubule assembly inhibition; m. inhibition of mitosis/microtubule organization; n. inhibition of cell division; o. inhibition of VLCFAs; p. inhibition of cell wall (cellulose) synthesis; q. uncoupling (membrane disruption); r. inhibition of lipid synthesis-not ACCase inhibition; s. action like indole acetic acid (synthetic auxins); and t. inhibition of auxin transport. In other embodiments, previously unknown metabolic states are identified as distinguished from known metabolic states associated with herbicide modes-of-action in an artificial neural network simulation.

In some embodiments, the biological samples are obtained from organisms of the same species. In various embodiments, the samples may be obtained from fungi tissue, a yeast tissue, bacteria, archaea, or animals such as insects, nematodes or mice for example. In other embodiments, the biological samples are obtained from plant tissue. More specifically, the plant tissue can be plant protoplast, whole plant, partial plant, callus tissue, or plant tissue of a cell suspension culture.

Therefore, the invention provides a method for determining the metabolic mode of action of a compound wherein said method comprises the method described above and wherein the subject biological sample is from an organism treated with the compound, and wherein the subject metabolic state indicates the metabolic mode of action of the compound. Alternatively, the invention provides a method for the determining the metabolic stress response in plants to stimuli wherein said method comprises the method described above and the subject biological sample is from an organism exposed to the stimuli, and wherein said subject metabolic state indicates the metabolic stress response to the stimuli.

The invention further provides embodiments of a metabolic profiling process wherein said process comprises: a. growing organisms under controlled conditions; b. treating a control subset of the organisms with known bioregulators; c. treating a subject subset of the organisms with an uncharacterized bioregulator; d. preparing samples of tissues of the subsets of the organisms; e. obtaining spectroscopic or chromatographic data of a plurality of metabolites from the samples; f. training an automated pattern recognition system by association of the spectroscopic or chromatographic data from the control subset of the organisms treated with the known bioregulator to determine a control metabolic profile; g. generating a mathematical model from the trained pattern recognition system based on spectroscopic or chromatographic data of the control subset of the organisms associated with the control metabolic profile; h. applying the mathematical model to the spectroscopic or chromatographic data of the subject subset of the organisms to determine the subject metabolic profile; and, i. comparing the subject metabolic profile to the control metabolic profile to determine the metabolic association of the uncharacterized bioregulator to the known bioregulator.

The invention further provides a metabolic profiling process wherein said process comprises: a. growing organisms under controlled conditions; b. selecting a control subset of the organisms with known phenotypic or genotypic traits; c. selecting a subject subset of the organisms with a potential unknown genetic modification or altered phenotype; d. preparing samples of tissues of the subsets of the organisms; e. obtaining spectroscopic or chromatographic data of a plurality of metabolites from the samples; f. training an automated pattern recognition system by association of the spectroscopic or chromatographic data from the control subset of the organisms to determine a control metabolic profile; g. generating a mathematical model from the trained pattern recognition system based on spectroscopic or chromatographic data of the control subset of the organisms associated with the control metabolic profile; h. applying the mathematical model to the spectroscopic or chromatographic data of the subject subset of the organisms to determine the subject metabolic profile; and, i. comparing the subject metabolic profile to the control metabolic profile to determine the metabolic association of the potential unknown genetic modification or altered phenotype to the known phenotypic or genotypic traits.

In some embodiments the genetic alteration comprises a gene mutation, gene deletion, or gene insertion. In some embodiments the genetic alteration comprises a gene activation change, such as a change in transcription factors, a change in promoters. In some embodiments the genetic alteration comprises a genetic modification such as a knockout of gene activity, inactivation of gene activity, or insertion of novel genes.

The invention further provides a database of metabolic responses comprising data generated from the above methods. These and many other embodiments will be apparent to one skilled in the art after a review of the entire description herein.

### DEFINITIONS

In this disclosure, a number of abbreviations and terms are used. The following abbreviations and definitions are provided:

As used herein, “a” or “an” means one or more than one depending upon the context within which it is used.

The term “Pattern Recognition” encompasses a series of methods in statistical analysis, which attempt to define a set of parameter values that will result in clustering objects with similar characteristics into regions of an n-dimensional space.

The term “Neural Network” is abbreviated “NN”. The term is used for a simplified, artificial model of the complex structure formed by neurons and their connectors: dendrites, synapses and axons. A NN can be defined as an interconnected assembly of simple processing elements (units or nodes, analogous to synaptic connections in the human nervous system) in a way which allows signals to travel throughout the network in parallel as well as serially. The processing ability of the network is stored in the inter-unit connection strengths (weights), obtained by a process of adaptation to, or learning from, a set of training patterns. Neural networks are an embodiment of a pattern recognition method. In the following, the term NN is used within the examples to represent a mathematical model, that includes all parts and methods needed to make it a tool useful to analyze data vectors, i.e. the term NN within the examples includes a particular topology, the methods used in the training and testing, and all weights, and activation values, functions, etc.

“Stress” is defined as any factor affecting an organism such as a pesticide treatment e.g. herbicide, insecticide, fungicide; deviating environmental factors, e.g. heat, light, temperature, air flow, level of water or nutrients, e.g. salts; addition or depletion of natural or unnatural compounds; lesions and other physical treatments; influence of bacteria, fungi, or animals, e.g. nematodes, insects; symbiotic and parasitic relationships which cause a positive or negative response in plant growth, health, tolerance or regulation.

A “Metabolic Response Database” is a database of spectra or chromatograms or data vectors derived from spectra or chromatograms, or patterns derived from such data vectors or derived from spectra or chromatograms, or mathematical models (neural network definitions) derived from such patterns, vectors, spectra, or chromatograms. Each entity in the database will be associated with the corresponding experimental conditions, treatments, samples sources and other relevant experimental information.

“Rescaling” a vector means to add or subtract a constant and then multiply or divide by a constant, as you would do to change the units of measurement of the data, for example, to convert a temperature from Celsius to Fahrenheit.

“Normalizing” a vector most often means dividing by a norm of the vector, for example, to make the Euclidean length of the vector equal to one. In the NN literature, “normalizing” also often refers to resealing by the minimum and range of the vector, to make all the elements lie between 0 and 1.

“Standardizing” a vector most often means subtracting a measure of location and dividing by a measure of scale. For example, if the vector contains random values with a Gaussian distribution, you might subtract the mean and divide by the standard deviation, thereby obtaining a “standard normal” random variable with mean 0 and standard deviation 1.

The term “metabolome” has been coined to describe the chemical profile or fingerprint of the metabolites in an organism. The metabolome reflects the life history of each individual plant, including age and environmental factors such as soil type and moisture content, temperature, stress factors, and exposure to applied fertilizers and crop protection chemicals. With the expectation that, following exposure to a herbicide, the herbicide's mechanism-of-action might be recognisable in the plant metabolome, we investigated whether such characteristics can be reliably detected in the NMR spectrum of a plant extract.

As described in the Background section, the gross chemical composition of various biological fluids has been investigated by a variety of chromatographic and spectral techniques, notably gas and liquid chromatography, NMR spectroscopy, mass spectrometry, and infrared spectrophotometry. In animal/human fluids, much of the NMR research has been directed towards disease characterisation and diagnosis. NMR has provided information on biosynthesis, and on the effects of herbicides on metabolism 21 and mode-of-action, or used in investigations of whole plants. A variety of computational methods have been applied for the statistical analysis of spectral data, including artificial neural networks. In many cases, however, it was found that environmental factors contribute significant “noise” to the metabolite profile and reproducibility has often limited the applicability. Furthermore, in many reports only two states (e.g. normal vs. treated) are simultaneously distinguished. A robust NMR method able to simultaneously detect multiple treatment groups has not previously been described. In the search for new pharmaceuticals and crop protection chemicals, it is sometimes desirable to have a fast and reliable means to detect the mode-of-action of a new active compound, or pinpoint unusual phenotypes by an altered metabolic profile. A practical method to accomplish this goal is provided by the present invention, and has subsequently been published, as Aranibar, N., Singh, B. K., Stockton, G. W., and Ott, K-H., “Automated Mode-of-Action Detection by Metabolic Profiling”, Biochemical and Biophysical Research Communications 286(1), 150-155 (2001).

There are currently established over twenty biochemical mechanisms for the numerous commercial herbicides used in agriculture (see Appendix I). We describe in this application the automated neural network analysis of ¹H NMR spectra of raw, aqueous plant extracts that can simultaneously, and with high reliability, detect the modes-of-action of the various herbicides. The computational classification utilizes artificial neural network methods that are shown to produce robust assignments under conditions where changes in sample characteristics are very small and often close to the statistical variation between samples.

The methods of the present invention are reliable when the experimental conditions are well controlled and accurately reproduced under standard conditions, for most herbicide modes-of-action. The present invention preferably uses optimized growing conditions, extraction procedures, and the bioanalytical methodology to produce highly reproducible conditions, thus creating a robust profiling method that is capable of detecting the many different herbicidal modes-of-action. Using only a small amount of tissue, the method is able to detect minute differences in a plant's metabolic profile even at an early stage of growth, where phenotypic changes are barely visible. The preparation and analysis procedures are simple and fast enough to permit screening of libraries of active compounds, with results being automatically and almost instantaneously reported, whereas traditional biochemical methods for mode-of-action determination require substantial experimental effort.

The present work has successfully demonstrated the simultaneous analysis in a single neural network nineteen MOAs that are established for the almost three-hundred herbicides used in agriculture, lending credence to the expectation that the method can be used to rapidly classify the herbicide mode-of-action for lead compounds in a routine NMR screen. Most important, the method can recognize when a new mode-of-action is present, which is considered extremely important for the herbicide discovery process.

In preferred embodiments, the present invention describes a metabolic profiling method for recognizing the state of biological, plant or microbial samples using spectroscopic and or chromatographic methods and pattern recognition techniques. The methods described herein comprise the steps of first selecting target organisms/plants and reference treatments, growing of controls and treated organisms under strongly controlled conditions, sampling of liquid isolates, using standardized chromatography/spectroscopy experiments to generate spectral response which correlates with a cellular state or bioregulator treatment. It further comprises of a pattern recognition method that allows us to classify the spectral response/metabolic profile with other similar spectral responses.

The method comprises of:

1. Growing selected organisms under controlled conditions while treating the organisms with known bioregulators or selecting organisms based on phenotypical/genotypical differences or employing various environmental stress factors.

2. Sampling of the biological tissue.

3. Generation of spectra or chromatograms from samples.

4. Optionally, building a metabolic response database.

5. Training or building of a mathematical model that is capable of associating the various treatments and coupling genetic differences, phenotypic differences or environmental factors with the metabolic profile of those organisms.

6. Application of mathematical models to spectra or chromatograms of the same or similar samples and detection of the metabolic profile of such samples.

7. Association of the metabolic profile with a treatment class

In the preferred embodiment, the treatment classes are first defined (in step 4), and the mathematical model is created to represent a database of known treatments (supervised learning methods). Such a mathematical model, as outlined in step 5, is applied to directly recognize the treatment classes.

Alternatively, treatment classes can be defined after detection of unknown treatment classes using suitable experimental techniques.

Selection of Target Organisms and Choice of Treatments

This step involves first selecting the target organisms. A series of reference treatments are performed on the target organism to define different cellular states corresponding to a particular treatment. For example, the correlation can be made between the compound treated, the specific organism, e.g. genetically modified organisms, and the specific response pattern which may include knockouts, expression of genes, and stress responses such as drought tolerance.

The target organism is selected according to the scientific or commercial interest. In a preferred embodiment it is an organism from one of the following groups: a crop plant, e.g. corn (Zea mays); a weed plant, e.g. wild oats (Avena fatua); a pest, e.g. rice blast (Magnaporthe grisea); and a model organism (e.g. Yeast, Synechocystis, Arabidopsis thaliana, C. elegans).

The choice of using one or more organisms, parts of an organism, the extraction method used or the time points of harvesting will depend on the question of interest and the analytical technique used. Persons skilled in the art will be able to select from the range of possibilities according to the suitability of the organism, tissue, or organism parts, the specific requirements and limitations of the various analytical techniques, and the expected information content existing in the metabolic profile of given samples and treatments. In the case of microorganisms, for example, a sample containing whole cells may be used to obtain NMR spectra of the metabolites within the cells. For plants, selection of a plant part that is known to be primarily affected by a given treatment can be sampled to increase sensitivity. For example, elongation tissue like growing points or young leaves are known to be largely affected by many herbicides.

Treatments are selected according to the interest of the study. In a preferred embodiment, treatment can be selected from the following groups: treatments with pesticides, employment of environmental stress factors, application of procedures to alter the activation of genes or the activity of gene products, or application of procedures to introduce genes, or alter gene products. All treatments usually include appropriate control samples. The use of a control herein is implicitly included by the term treatment, i.e. controls are only specific forms of treatments.

In another embodiment, samples from a species are selected that have characterized or uncharacterized gene alterations, genetic modifications, or altered phenotypes. For example, seeds from corn that has or lacks resistance to herbicides or pests can represent a selection of samples.

In another embodiment, the selection of treatments is chosen to represent a set of predefined conditions to establish a knowledge base of treatment/response patterns for a wide variety of biochemical pathways or environmental stress factors of interest. For example, there are currently 28 known modes-of-action classified for herbicides. Each class is represented by one or more herbicides. A database of metabolic profiles of herbicidal modes-of-action can be built by selecting one or more herbicides from each class, and using them in above described method. Similarly, a selection of organisms resistant, tolerant or sensitive to a pesticide or pest can be used to create a metabolic profile database. For example, imidazolinone sensitive, imidazolinone tolerant and imidazolinone resistant plants (seeds) can be selected to create a metabolic profile database for alterations in the ahas gene and the branched-chain aliphatic amino-acid pathway, because imidazolinones inhibit the AHAS protein which catalyses the key step in the valine, isoleucine, and leucine biosynthesis pathway.

Growing Conditions

The organisms selected for treatment are grown under controlled conditions, where the conditions are all external factors that can be regulated e.g. temperature, timing, supply of nutrients, and for which a change in conditions may produce modifications in the metabolic profile of the organisms. Treatments are varied but are applied under conditions that are also strongly controlled and that minimize variations as much as possible.

It is critical to maintain highly controlled, reproducible growing conditions because even small changes in environmental or other factors may lead to changes in the metabolic profile. Such changes may obscure the changes caused by the chosen treatment. The need to control growth conditions accurately appears to require more stringent controlled conditions that those usually applied for screening purposes. Plants are grown under standardized conditions with controlled water and supply of nutrients in commercial growth chambers where there is full control over light, temperature, and humidity.

For example, corn (Zea mays) seeds (Pioneer 3514) were set to germinate in paper towel rolls in tap water covered with plastic foil (to minimize evaporation) for 5 days in the growing chamber. Conditions were adjusted to “summer days” (day/night 14/10 hours, controlled temperature 27° C. and humidity 70%). After germination the seedlings were visually inspected. Seedlings that were homogeneous in size and appearance were selected and set to grow in hydroponic Hoagland culture solution.

Each seedling was set in a 50 mL dark bottle in 25 mL Hoagland nutrient solution. The plants were then grown for 5 more days after they had reached the three-leaf stage. At this point the hydroponic solution was changed and the seedlings were treated as follows:

Different herbicide stock solutions in acetone were added in concentrated form to the hydroponic solution or, in the case of control plants with 20 μL acetone, or

The hydroponic solution was replaced by a solution containing different concentrations of herbicides, or just hydroponic nutrient solution for control plants.

All herbicides were technical grade. The plants were returned to the growing chamber after treatment. After 24 hours, the plants were harvested by excising between the coleoptile and the first leaf collar. The first leaf sheet was separated and the meristematic tissue collected was flash frozen in liquid nitrogen in a cryogenic 3 ml tube and stored in the liquid nitrogen freezer until further use.

Sampling of Liquid Isolates from Biological Tissues

Liquid isolates, which can include aqueous or organic extracts of cell lysates from the target organisms, or suspensions of partial or whole organisms, e.g. microbia, can be sampled manually or robotically according to standard procedures known in the art.

For example, frozen meristematic tissue was placed in a mortar and liquid nitrogen was added. The pestle was also allowed to cool in the liquid nitrogen. When the liquid nitrogen was evaporated, the plant tissue was pulverized in the mortar. Then, 2.4 mL of 0.25N aq. HCl solution were added to the mortar and the sample was further mixed with the pestle. The suspension was placed into an Eppendorf centrifuge tube and set in ice until all the samples for an experiment were processed for centrifugation. The samples were centrifuged at 14000 g, at 4° C., for 60 minutes. The supernatant was separated from the pellet and 0.8 mL taken and mixed with 0.2 mL D₂O (with TSP 0.05 w/v for NMR reference) for the lock signal in the spectrometer. The samples were kept in ice until NMR measurement.

Generation of Spectra or Chromatograms from Samples

Standardized chromatography/spectroscopy experiments (e.g. NMR, MS, Flow-NMR, LC-NMR, Flow-MS or LC-MS) to identify specific chromatographic responses to treatments of target organisms are the preferred means of creating a profile of the metabolite mixture of the samples. It is important that the experiments are performed in a highly reproducible manner for all samples that are being compared, classified, or clustered. Also, all samples that are being classified need to be treated and processed under the same conditions as the samples that are used to establish the mathematical models for classification.

The data acquired and processed on the analytical instrument is exported and converted into a format suitable for the ANN program used. Usually, the spectral information is in the form of a series of vectors with intensities. The JCAMP-DX format was used as a common, intermediate format that can be exported from most analytical instruments.

Example of standardized experimental conditions for NMR spectra generation:

The proton NMR spectra of plant extracts were recorded using a Bruker AMX 500 NMR spectrometer equipped with a TXI 5 mm probe. The probe temperature was carefully regulated to better than ±0.1 K using the Bruker/Haake variable temperature accessory, and all spectra were recorded under identical experimental conditions, as follows:

TABLE 1
Standardized NMR Acquisition Parameters

| Parameter | Setting |
|---|---|
| Pulse program: | zgpr (solvent presaturation at center |
| | frequency) |
| Time domain: | 16384 points (complex points) |
| Number of scans: | 256 |
| Number of dummy | 256 (i.e. 10 min for temperature equilibration) |
| scans: | |
| Temperature: | 295.0K |
| Spectral width: | 5555.56 Hz |
| Acquisition time: | 1.47461 sec |
| Water saturation pulse | 1 sec at 60 dB |
| Acquisition Pulse | 4 μsec (@ 3 dB equivalent ˜ 45° pulse width |
| Transmitter Frequency | 500.1323559 MHz |

Example of Standardized NMR Spectra Preprocessing

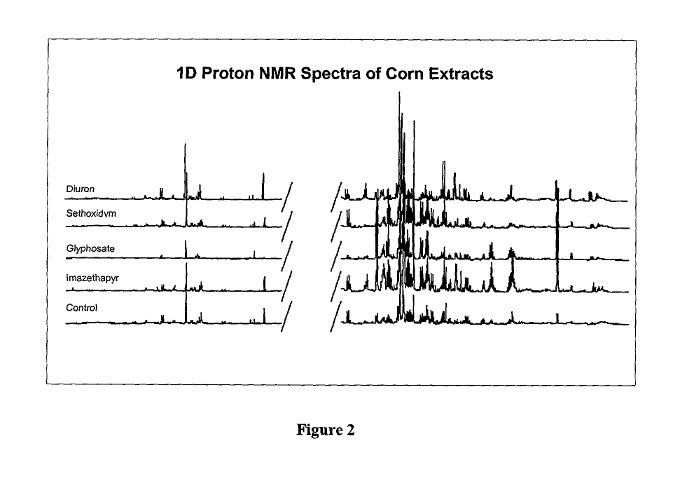

The NMR spectra were multiplied with an exponential function (LB parameter=0.5 Hz), Fourier transformed, and manually phase- and baseline-corrected. Spectra were, in an automatic fashion, exported into JCAMP-DX format and converted into pattern vectors for pattern recognition approaches. A window of points was removed from the central part of each vector prior to analysis, to avoid the water residual signal as shown in FIG. 2. Also, data points were removed at the low field and high field portions of the spectral vector because no resonance signals were detectable in these regions.

Similar procedures can also be applied to any other spectroscopic or chromatographic technique that produces a profile for a sample in a form that can be converted into a data vector or matrix. These procedures may include resealing, normalization or standardizing of the data vectors or matrix. The conversion might, also, include suitable data reduction and scaling steps. In the present invention, where the dominating solvent signals were removed, normalization and scaling of the spectra was possible. Scaling the spectra to a mean value of 1 provided good results. There is ample discussion of other averaging and scaling methods in the literature.

For some spectral techniques, like NMR, it is usually advisable to eliminate parts of the spectrum that contain signals that have limited information contents e.g. large solvent or buffer signals. For example, in the NMR spectra we have eliminated a region of about 2 ppm (parts per million of the frequency spectrum) that contain the water resonance, when using aqueous extracts. Further preparation of the input vectors includes scaling of the spectra to remove the amount of divergence between spectra and reduce the number of necessary training sets. Scaling the spectra to a mean value of one (1) avoids also very large or small intensity values thereby reducing the problems associated with round-off artifacts in the computer. Scaling can be performed using a reference signal intensity, e.g. a fixed amount of TSP that is usually added to the NMR sample for internal reference, or the overall intensity of the spectrum, e.g. each spectrum has been scaled to a mean intensity of 1. Scaling can also be achieved by methods provided by the NMR analysis software used for processing the spectra. Many similar methods are described in the literature. Alternative methods are advisable when one or more very large signals e.g. from solvents or salts, are present in the spectra.

It is also possible to re-digitize the data to decrease the number of data points or adjust for changes in spectrometer frequencies or similar, and to decrease the required computational time. For example, it was found that from a spectrum with 8 k data points every 5 points may be binned into one datapoint without loosing significant informational content. The analysis of the NMR spectra had shown that a typical resonance line is defined by more than 5 data points. Therefore, it was concluded that only some signal resolution would be lost in very crowded regions of the spectra, but at the same time compensate for this by a gain in sensitivity. Such binning steps are mostly unnecessary given a ready availability of fast computer workstations, except for a thorough, systematic analysis of training conditions or similar where computational time might become an issue.

Generation of a Metabolic Response Database

In a preferred embodiment, the invention describes a metabolic response database developed from bioregulator treatments, specific gene modifications and interruptions in metabolic pathways, which induce positive or negative responses in spectral components. This involves the generation of a database of information that contains, for specific defined treatments, the metabolic profiles in a suitable format. The metabolic response database is used to capture the spectra, chromatograms, data vectors, patterns and/or mathematical models (e.g. neural networks) which are used to identify corresponding treatments, or gene, genetic, or phenotypic alterations. The database includes, for each sample, the description of the treatment for that sample, and at least one of the following: the spectra and/or chromatograms from that sample, a data vector, or a pattern definition derived from the spectra and/or chromatograms. The database may be implemented within a relational database scheme by itself, or as part of a laboratory information system, or in form of a computer file system database, i.e. an organized storage of the data files. For example, the current 28 classes of known herbicidal modes-of-action can be represented by a metabolic database by selecting one or more herbicides from each class, growing organisms under controlled conditions, and applying such herbicides to individual samples of such organisms. The treatment information and the corresponding spectra and/or chromatograms of each sample are then collected and stored in a suitable database. It is within the scope of the invention to apply those techniques alone or in combination to plants, fungi, insects or microorganisms.

Profiling Methods

Profiling methods encompass techniques that analyze experimental information from a series of samples to derive knowledge about elements that are representative for a given treatment. Such knowledge is encoded usually in a mathematical model e.g. neural network. If an experiment done on a sample produces a pattern of representative elements very similar to a previous sample, it is likely that the new sample has similarity to the previously known sample. Standard statistical methods are used to estimate the degree and significance of the detected similarity. The profiling methods do not rely on a selection of signals, reporter compounds, or similar to represent a treatment of cellular state. In contrast, a profiling method uses the experimental information as a whole to derive, using mathematical/statistical approaches, representative patterns for each group. The algorithm derives such patterns, hence the patterns are not based on a user selection. The strength of the profiling methods relies on the fact that all or most of the experimental knowledge is used in a correlated fashion, thus maximizing the use of the information contents of the data. The profiling method described herein also does not require laborious and expensive previous separation of the sample in its components, making it suitable for higher throughput and increasing the robustness of the approach.

The present invention describes in preferred embodiments a NN designed to utilize the metabolic response database to detect metabolic changes in microorganisms and/or plants then correlates such spectral response with a cellular state or treatment. The theory of NN teaches that there are two general classes of NN approaches. One class encompasses methods that use a supervised learning scheme in which patterns are presented to an untrained network together with the expected output activation values. A training of the network is performed to adjust the weights of the connections to match the input vector with the activation of the output nodes (“training step”). The resulting trained NN is then used to classify the same or other samples during the “testing step.”

The second class of NN approach is based on unsupervised learning and does not require a training step. This NN approach, however, classifies groups of input patterns without prior knowledge of the class definitions and without relating and comparing them to one another.

The NN analysis is made using NN simulators. A wide variety of commercial, freely available or home-written programs can be used. In the preferred embodiment the SNNS (Stuttgart Neural Network Simulator) package that offers flexibility and throughput has been applied. The program package has been augmented with an additional set of research tools (programs and scripts) that perform a variety of automation tasks that are described and exemplified below.

The NN approach requires the definition of a neural network architecture that matches the learning scheme (supervised/unsupervised), the type of algorithm (e.g. feed-forward, backward propagation), and the size of input and output vectors.

Definition of a Network Architecture

Exemplified here is a NN topology that is appropriate for a supervised, backward propagation learning. This topology must have a number of input nodes that corresponds to the number of data points of a single input vector. In the most common approach, a 3-layer ANN with an input layer that represents the spectral information, one or more hidden layers, and an output layer that has one node for each group to be classified is used. The connections between the layers is complete without any shortcuts, i.e., each input unit is connected to each hidden unit, each hidden unit is connected to each output unit. All connections are directed from the input toward the output (“feed-forward network”). The number of input nodes has to match the number of spectral data points that are to be considered for the ANN analysis. The sampling of most of the frequency response with at least one point per individual resonance line (for proton NMR) yields good results. More points become advantageous as the database grows.

For example, if 5000 data points from the NMR spectra have been selected, the length of the input vector is 5000. It also requires output nodes that indicate the type of treatment group. The number of output nodes needs to correspond to the number of treatments that are encoded in the output node vectors, e.g. six in the example described above. The number of hidden layers is variable and should be large enough to sensitively encode the spectral information content. We describe, in the example section, an experiment that indicated that 12 hidden units are sufficient to encode at least 71 different experiments that are strongly related. The number of hidden units appears to be less significant for a successful approach. Theoretically, any number of hidden units is allowed, a reasonable range would be from zero (0) i.e. no hidden layer to the number of input nodes. It is of course possible to use multiple layers of hidden nodes. However, this appears to be not necessary for the approach outlined herein. It might become useful if a large number of different treatments need to be encoded.

Providing a Set of Input and Corresponding Output Vectors for Training of the Network

The method of training, validating, and using a NN includes steps to export and convert the spectral information into a format suitable for reading by the neural network simulator program. In most cases, the software used to analyze the spectral information from the analytical instrument, e.g. the NMR spectrometer software, is equipped with routines to export the processed spectral information in the form of an ASCII-formatted file. In the preferred embodiment, the spectra are exported in a standardized format like the JCAMP-DX format (Joint Committee on Atomic and Molecular Physical Data Exchange References). For example, the XWinNMR program function TOJDX (Xwin-NMR User Manual, Bruker Spectrospin GMBH, Karlsruhe, Germany) converts spectra into the standard format JCAMP-DX. From this intermediate format, the data values for the input nodes are extracted by a suitable computer program that can be generated by any person skilled in the art and written in a format that the NN program can read. During this step, it is also possible to select the regions of interest that are to be included into the input vector. For example, it is advisable to exclude a large solvent resonance in the NMR spectrum, like the resonance signal of the water protons from the input vector. Regions with little or no information can also be excluded. However, it is important to keep processing for training, validation, and testing sets in common for use as input vectors (patterns) by a single NN.

It is also necessary for the training set (and advisable for the validation set) to define the values for the output nodes if a supervised learning procedure for the NN is to be used. The number of output layer nodes is matched to the number of states that are to be classified, i.e., for each treatment class an output node is defined. For each input vector of the training or validation set that represents a given cellular state (i), the i-th element (or node) of the output vector is set to one (1) while all others elements are set to zero (0), yielding a corresponding output vector for each input vector.

For example, in some of our examples described in more detail elsewhere in the present invention, six states have been defined corresponding to a control, 4 different herbicide treatments, and a state for diseased plants. Therefore, we needed to define at least six output nodes (output node 1-6, respectively). For the training set (and the validation set) an output node was set to 1 or 0 to indicate whether a sample represented or did not represent the respective treatment, i.e. to indicate a Control, the output node 1 was set to 1, the remaining output nodes for this pattern were set to 0. Similarly, the PURSUIT® (imazethapyr) treatment was indicated by the second output node being set to 1 and all the other output nodes (1 and 3-6) set to zero.

Additionally, each vector in the series can associate textual information that traces the origin and history of the sample. For data vectors that are being used as part of the training set, the information for the “output node” of the NN has also to be provided for each individual data vector. Each element of the vector of output nodes represents one group of treatments, e.g., branched-chain amino acid biosynthesis inhibitors. The output vector corresponding to each input vector thereby usually contains a ‘1’ (one) setting for the element that represents the treatment that spectral data vector represents, and ‘0’ (zero) if the input vector is part of the training or validation set. The output vector is undefined at first for input vectors that are to be recognized (test sets).

The validation set is labeled in a similar way. A computer program can, after testing of a NN, read the program output and create a report that indicated the correctness and failures of the NN for each particular experiment. A partial example of such a file, named pattern file in the following, is shown below. Comments in brackets are not part of the file but indicate values being removed for clarity and brevity. Further information about valid file formats is to be taken from the software documentation of the NN simulator that is being used.

SNNS pattern definition file V4.1

generated at Thur Mar 16 08:16:06 EST 2000 Ranges: 965 3440 4330 7254. Bin-Size: 5.

Scaled to Mean of 1

No. of patterns: 71

No. of input units: 1080

No. of output units: 6

# na022400_{—}01 1: Control

−0.0172958965286963 0.00651549589855155 0.00180059827977478 . . .

[ . . . a total of 1080 data values for the input vector 1 . . . ]

0.00629101465956216 0.00763400457292774

# na022400_{—}01: pattern Control

1.000 0.000 0.000 0.000 0.000 0.000

. . . [next records describing the remaining input and output nodes as lines # na0220400ff]

Pattern Recognition Using Neural Networks with Supervised Learning

The NN approach using a supervised learning scheme requires training of an artificial NN or similar pattern recognition methods to correlate spectral response with a cellular state or treatment:

Steps include:

8. Providing a set of input and corresponding output vectors for training of the network.

9. Training the appropriate network topology using appropriate algorithms.

10. Presenting of input vectors to the trained network for validation

11. Presenting of input vectors to the trained network for classification.

An important focus of neural network research is how to adjust the weights of the links to get the desired system behavior. This modification is very often based on the Hebbian rule, which states that a link between two units is strengthened if both units are active at the same time. For example, training a feed-forward neural network with supervised learning consists of the following procedure:

12. An input pattern is presented to the network;

13. The input is then propagated forward in the net until activation reaches the output layer. This constitutes the so-called forward propagation phase; and

14. The output of the output layer is then compared with the teaching input. The error, which is the difference (delta) between the output and the teaching input of a target output unit ‘j’, is then used together with the output of the source unit ‘i’ to compute the necessary changes of the link. To compute the deltas of inner units for which no teaching input is available, (units of hidden layers) the deltas of the following layer, which are already computed, are used. In this way the errors (deltas) are propagated backward. This, therefore, constitutes the so-called backward propagation phase.

In on-line learning i.e. after each forward and backward pass, the most widespread learning algorithm is currently “backpropagation”. Backpropagation works by changing the weights of the connections after each training pattern. There are several other algorithms that differ in properties like speed, sensitivity and robustness. The training is usually halted either by setting the number of training cycles in advance or by training the network until it has reached a predefined error on minimum for the training set or, better yet, the validation set.

One of the major advantages of neural nets is their ability to generalize. This means that a trained net could classify data that it has never seen before where the new data is from the same class as the data used for training the net. In the present invention only a small part of all possible patterns for the generation of a neural net is available. For example, we can train the network with spectra obtained by treating a plant with PURSUIT® herbicide. The network should later recognize plants treated with another branched-chain amino acid biosynthesis inhibitor belonging to the same class as the PURSUIT® herbicide.

In order to achieve the best generalization, the data set should be split into three parts:

15. The training set is used to train a neural net. The difference between the predefined output node value and that produced by the network for each pattern (the error) is minimized during training.

16. The validation set is used to determine the performance of a neural network on patterns that are not used for training during learning. To avoid overtraining the error level of recognizing inputs of validation set is often used to determine the end of the training cycles. (Overtraining refers to a phenomenon that is often seen during the training of neuronal networks. The algorithm is tailored to minimize the error on the training set. However, while doing so, there exists a change to loose generalization by encoding features from the training set that are of statistical nature (see Step 3 for methods to deal with overtraining).

17. A test set for finally checking the overall performance of a neural net or the real world application.

The learning should be stopped in the minimum of the validation set error. At this point the net generalizes best. When learning is not stopped, overtraining may occur and the performance of the net on the whole data decreases despite the fact that the error on the training data still gets smaller. After finishing the learning phase, the net should be finally checked with the third data set, the test set. This methodology is referred to as supervised learning since it teaches the network with a pattern of known output.

Algorithms

The learning method found to yield reliable results under a wide variety of training conditions, fast convergence, and classification with minimum error was Resilient Backpropagation (SNNS User Manual, University of Stuttgart, and A. Zell “Neuronal Networks”). This function is known in the literature to produce consistent, robust and fast learning with good generalization. The basic principle of resilient back-propagation in the Rprop module is to eliminate the harmful influence of the size of the partial derivative on the weight step. In consequence, only the sign of the derivative is considered to indicate the direction of the weight update. The size of the weight change is exclusively determined by a weight-specific, so called “update-value” Δᵢⱼ^{(t)}. In addition, a weight decay parameter α determines the relationship of two goals, namely to reduce the output error (the standard goal) and to reduce the size of the weights (to improve generalization). Adjustment of the weight decay factor can become necessary if it is observed that the overtraining occurs and more generalization is desired. Smaller values on the weight (2-4) lead to slower convergence but better generalization.

The composite error function is:

E=Σ(t_{I}−oᵢ)²+10^{-α}Σωᵢⱼ²

The size of the weight change is determined by:

$$Δ {ϖ}_{ij}^{( t )} = ( \begin{matrix}- {Δ}_{ij}^{( t )} : & if \frac{\partial {E}^{( t )}}{\partial {ϖ}_{ij}} > 0 \\ + {Δ}_{ij}^{( t )} : & if \frac{\partial {E}^{( t )}}{\partial {ϖ}_{ij}} < 0 \\ 0 : & else\end{matrix}$$

Where Δ\overline{ω}ᵢⱼ^{(t)} denotes the summed gradient information over all patterns of the pattern set (“batch learning”).

The second step of Rprop learning is to determine the new update values Δᵢⱼ^{(t)}. This is based on a sign-dependent adaptation process.

$${Δ}_{ij}^{( t )} = ( \begin{matrix}{η}^{+} \pm {Δ}_{ij}^{( t - 1 )} : & if \frac{\partial {E}^{( t - 1 )}}{\partial {ϖ}_{ij}} \pm \frac{\partial {E}^{( t )}}{\partial {ϖ}_{ij}} > 0 \\ {η}^{-} \pm {Δ}_{ij}^{( t - 1 )} : & if \frac{\partial {E}^{( t - 1 )}}{\partial {ϖ}_{ij}} \pm \frac{\partial {E}^{( t )}}{\partial {ϖ}_{ij}} < 0 \\ {Δ}_{ij}^{( t - 1 )} : & else\end{matrix} \\ where 0 < {η}^{-} < 1 < {η}^{+}$$

The adaptation rule works as follows: every time the partial derivative of the corresponding weight ωij changes its sign, which indicates that the last update was too big and the algorithm has jumped over a local minimum, the update value Δij(t) is decreased by the factor η−. If the derivative retains its sign, the update value is slightly increased in order to accelerate convergence in shallow regions. Additionally, in the case of a change of sign, there should be no adaptation in the succeeding learning step. In practice that can be achieved by setting δE^{(t-1)}/δwᵢⱼ to 0 in the above adaptation rule Rprop tries to adapt its learning process to the topology of the error function; it follows the principle of “learning by epoch”. This means that weight update and adaptation are performed after the gradient information of the whole pattern set is computed. The Rprop algorithm takes three parameters: the initial update value Δ⁰, a limit for the maximum step size, Δₘₐₓ and the weight decay exponent α.

A robust and widely applicable set of parameters, as shown in Table 2, has been derived empirically starting with values known from the literature.

TABLE 2
Preferred Parameters for SNNS

| Parameter | Value |
|---|---|
| Learning function: | Resilient Back Propagation |
| Update function: | Topological Order |
| Initialization Function: | Randomize Weights between −1 |
| | and 1 |
| Initial update value: | 0.1-0.5 |
| Weight decay exponent | 4-9 |
| Maximum step size: | 50 |
| Number of layers: | 3 (1 input, 1 hidden, 1 output) |
| Input layer: | 1080 nodes (Example only) |
| Hidden layer: | 12 nodes |
| Output layer: | 6 nodes (Example only) |
| Activation function: | Logistic (unbiased) |
| Output function | Identity |

Activation Function

The activation function is part of each neural network unit. It determines the activation value of a unit as a function of the sum on input values to that unit. In some networks a specific output function is also defined, usually the output function is the unity function operating on the result on the activation function.

Update Function

The update function determines the specific sequential order that the neurons are visited in order to perform operations on them. This order depends on the topology of the net and influences the outcome of a propagation cycle. The topological order update function that has been used in the given examples is the most favorable for feed forward nets. The neurons calculate their new activation in a topological order. This means that the first processed layer is the input layer, the second one is the first hidden layer, and the last one the output layer.

Initialization Function

A specific function is required that initializes the components of a net. Backpropagation, for example, will not work properly if all weights are initialized to the same value. The function used “Randomize Weights”, initializes all weights and the bias with distributed random values. The values are chosen from the interval (a, b), where it is required that a>b.

Detection of Treatment Class

Metabolic pathways affected by a treatment are identified by spectral components for which reference treatments have established a representative pattern. If significant portions are in match, between reference and unknown or other groups of samples, it is most likely that such treatments have the same or very similar effect onto the metabolic profiling.

The identification of the metabolic pathway affected can also be determined from analysis of the metabolic spectral components. The spectral components for which novel metabolic pathway inhibitors induce a positive or negative response are specifically identified. Such responses thereby identify the pathways or pathway components that are affected.

Detection of the cellular state or treatment class through the neural network is achieved by presenting spectra in the form of a pattern to the neural network, as described above for the training set, with the exception that the NN is not further changed but the response activation values of the output nodes are recorded for each spectrum presented. If the activation value of one of the output nodes is high i.e. >0.7 but usually >0.95-1.00, that particular spectrum is classified as similar or identical for activation values >0.95, to the group which is represented by the output node that exhibits a large activation. Such values have been established in the art. In the present invention, the following definitions are used to provide a more rigorous classification that highlights false assignments for the purpose of method evaluation and validation: Samples are assigned to a group if the corresponding activation value of the output node is >0.7 and no other node is >0.4. In praxis, one might choose that the former value to be larger, and the latter value to be smaller to decrease the change of false positives. Such values are adjustable by persons skilled in the art, and the particular choice will need to be established by experimentation as described in our example section.

Intermediate values or activation of several output nodes simultaneously indicates problem cases that are not yet represented in the database and may indicate a novel mechanism for that particular compound.

Pattern Recognition Using Neural Networks with Unsupervised Learning

The profiling methods can also be applied in the same way as described above, but without prior building of a database from samples with predefined treatment classes. The method would then be applied in a way by which the metabolite profile would be presented to the neural network that has been trained only with control samples. Deviation from the control spectrum would thereby indicate a genetic modification or other treatment that affects the metabolic composition. This approach would be preferred for example, as a high-throughput primary screen to detect the effect of a genetic modification of activity of a newly introduced genetic element (gene insert, knock-out transformation, etc.) or of a treatment with a possibly very weakly bioactive/pesticidal compound.

While unsupervised learning can be advantageous for some applications, in particular for the screening of genetic modifications of organisms, the supervised learning method which uses ANN technologies to classify groups of inputs (“cluster”) is preferable for the screening of large numbers of genetically modified organisms. If an abnormal pattern is seen, the function of one or more representatives of the cluster can be determined by homology. Conclusions about the physiological effect of such genes will enable targeted design of additional characterization either by other functional genomics approaches or by creating reference samples in the way described above to determine in more detail the function of the members of that cluster.

### Utility

The invention provides functional genomics capabilities and allows mode-of-action studies. It supports and complements other functional genomics or mode of action methods. Its major advantage is that it can detect small changes in the composition of metabolites that could otherwise only be detected using sophisticated separation methods, combined with extensive applications of analytical techniques to identify each component.

The method can be used to identify the metabolic pathways that have been up- or down-regulated in genetically modified plants.

The methods of this invention can be used to determine the mode of action of a new herbicide or lead compound.

The methods of this invention can be used to determine and compare the genetic profile of genetically modified plants.

The methods of this invention can be used to determine the influence of stress factors in plants/microorganisms as deduced from their metabolic response. Stress factors include any factor such as a pesticide treatment e.g. herbicide, insecticide, fungicide; deviating environmental factors, e.g. heat, light, temperature, air flow, level of water and other nutrients, e.g. salt; addition or depletion of natural or unnatural compounds; lesions and other physical treatments engaging of bacterial, fungal, or animals, nematodes, insects; symbiotic and parasitic relationships which causes a positive or negative response in plant growth, safety, tolerance, regulation or production. These stress factors may also be linked to the metabolic responses to gene and genetic alterations and modifications. Such modifications include, but is not limited to, gene mutations, gene deletions, gene insertions, gene activation changes such as change in transcription factors or change in promoters or change in vectors; and genetic modifications such as knockout of gene activity or inactivation of gene activity and/or repression of genes by oligonucleotides or modified oligodeoxynucleotides.

Additionally, methods of this invention can be used to compare the profile of protein expression with the protein product in genetically modified plants/microorganisms. The profile of protein expression can be correlated with the metabolic responses to stress factors.

The methods also find utility in the screening of biologically active compounds including fungicidal, herbicidal, insecticidal and nematicidal compounds. The particular screening methods include primary and secondary screens typically used in the discovery of new pesticides. The methods enhance mode of action determinations by linking mechanisms of action to specific metabolic profiles thus providing HighThroughPut means for the screening of compounds for fungicidal, herbicidal, insecticidal or nematicidal activity.

### EXAMPLES

The sample preparation is fast, simple and low in cost in comparison with other techniques. It requires one purification step. All steps can be automated and a high throughput can be achieved making this a method for high throughput screening of therapeutic or pesticide leads as well as genes. The automated analysis using neural network or similar pattern recognition techniques is extremely sensitive, robust and fast.

### Example 1

### Experiments to Validate the Neural Network Approach

In order to evaluate method validation, this example investigated whether

[1] Spectra from different treatments can be significantly different such that a NN can distinguish between them;

[2] Changes related to treatments can be large enough to allow robust distinctions between treatments;

[3] Changes between individual samples of the same treatment can be small enough to not disturb recognition or changes unrelated to treatment can be incorporated into network training to be recognized by the NN as such;

[5] Similar treatments really produce similar spectra i.e. can the network generalize to such groups as specific mode of action.

All the examples here are based on NMR spectra from corn seedling extracts.

A first set of 71 spectra, with 3 batches of 6-9 control samples, 2 batches with 15 PURSUIT® treated samples, and one batch each with 6(4) Sethoxydim, Glyphosate, and Diuron treated samples. Two plants fouled after the herbicide treatment phase, and were treated as separate category, exemplifying samples with very different properties.

The neural network topology used is based on a fully connected, three layer backward propagation network, as described below in the example section.

### Example 2

### Sensitivity of the Neural Network

To establish sensitivity sets of computer experiments were performed with various selections of spectra for training and validation of the network:

The network was trained with all 71 spectra described above. The spectra were then presented again to the trained network as test samples. All 71 spectra were individually recognized. This indicates that the NN is very sensitive to detect even very small changes like those between replicates. It also indicates, that the network topology chosen (i.e. a three layer network with 12 hidden units) is capable of encoding at least 70 different output nodes even if the inputs are very similar. This network topology was adopted for all further tests. The test proved furthermore that the chosen activation function settings, and other parameter settings are adequate for our approach. A survey of various training functions, and their parameters has also been performed. The results are summarized below. While almost all methods and a wide range of parameters yield acceptable to excellent results, preferable is the Resilient Backpropagation [Riedmiller, M., Proceedings of the SNNS 1993 workshop, Riedmiller, & Braun, Proceedings of the IEEE International Conference on Neuronal Networks 1993] with the following parameter settings: Delta starting values for all Δij (default value is 0.1): 0.5, practical range: 0.01-0.9. Delta(max), the upper limit for the update values default and preferred value is 50. This parameter is not critical for success of the training. α, the weight-decay, determines the relationship between the output error and to reduction in the size of the weights. In SNNS, the weight decay parameter denotes the exponent of the error decay exponential function e.g. the default of 4 corresponds to an error decay of 1:10000. Values between 2 and 9 are preferred.

### Example 3

### Conditions for Production of Neural Networks with High Recognition Potential

Typically, a selected or randomly chosen group of spectra is used to train a network. The remaining spectra from a group of experiments can be used to validate the network.

Using 30-40 spectra chosen that way and the remaining spectra (out of 71) for validation, it was found that in general, any set of training spectra yielded fall recognition of the validation set if at least one spectrum for each batch and at least two spectrum for each treatment/control were included in the training set. If the experimental conditions are kept constant, two or more spectra representing each treatment are sufficient to produce a sensitive NN that can recognize other samples of the same treatment, without the necessity to include samples from each batch.

### Example 4

### Creation of a Robust and Sensitive NN, and Definition of a Full Training, Validation, Testing Cycle

The following describes a complete experiment:

As described before, 15 spectra, out of 71, were selected for training the NN. The NN recognizes all remaining spectra with high confidence. The following a list of steps to be taken:

18. Untrained pattern loaded. (see Table 3)

19. Learning function is Rprop. Parameters are: 0.5, 50, and 9

20. Init. function is Randomize_Weights. Parameters are: 1, −1

21. Update function is Topological_Order

22. Net initialized

23. Cycles trained: 175 to reach convergence of 10e-9.

24. Analysis: Total Error on training set: 8.49682e-08

25. Patternset sel4c.pat loaded; (see Table 4)

26. Statistical Analysis(56 patterns Net i1080h12o6 net loaded

27. Patternset: sel4.) Wrong: 0.00% (0 pattern(s));

28. Right: 100.00% (56 pattern(s))

29. Unknown: 0.00% (0 pattern(s)) total error: 0.0032

TABLE 3
Training set “sel4.pat”. The spectra listed in this
table in column 1 have been converted into patterns and were
presented to the network as described below. The output
nodes were set to indicate the Treatment (2^{nd} column).

| Spectrum designation | Treatment |
|---|---|
| Batch 022400_03 | Control |
| Batch 022400_05 | Control |
| Batch 022400_16 | PURSUIT ® |
| Batch 022400_20 | PURSUIT ® |
| Batch 030100_03 | Control |
| Batch 030100_05 | Control |
| Batch 030100_08 | PURSUIT ® |
| Batch 030100_11 | PURSUIT ® |
| Batch 030600_08 | Sethoxydim |
| Batch 030600_09 | Sethoxydim |
| Batch 030600_13 | Foul |
| Batch 030600_16 | Glyphosate |
| Batch 030600_17 | Glyphosate |
| Batch 030600_21 | Diuron |
| Batch 030600_22 | Diuron |

TABLE 4
Validation_set: sel4c.pat. The spectra of listed in this table were con-
verted into patterns in the same way as those of pattern sel4.pat.
Presenting these patterns to the network trained as described below
with pattern sel4.pat resulted in an output node activation that is
translated into the assignments shown in the Network recognition
column (activation values were all >0.99). For a comparison, the
actual treatment the samples were subjected to are listed in Treatment.
It is thereby demonstrated that this network has recognized all 56
samples of the validation set correctly.

| Spectrum | | | Network |
|---|---|---|---|
| Designation | Spectrum number | Treatment | Recognition |
| Batch 022400_01 | 1: | Control | Control |
| Batch 022400_02 | 2: | Control | Control |
| Batch 022400_04 | 4: | Control | Control |
| Batch 022400_06 | 6: | Control | Control |
| Batch 022400_07 | 7: | PURSUIT ® | PURSUIT ® |
| Batch 022400_08 | 8: | PURSUIT ® | PURSUIT ® |
| Batch 022400_09 | 9: | PURSUIT ® | PURSUIT ® |
| Batch 022400_10 | 10: | PURSUIT ® | PURSUIT ® |
| Batch 022400_11 | 11: | PURSUIT ® | PURSUIT ® |
| Batch 022400_12 | 12: | PURSUIT ® | PURSUIT ® |
| Batch 022400_13 | 13: | PURSUIT ® | PURSUIT ® |
| Batch 022400_14 | 14: | PURSUIT ® | PURSUIT ® |
| Batch 022400_15 | 15: | PURSUIT ® | PURSUIT ® |
| Batch 022400_17 | 17: | PURSUIT ® | PURSUIT ® |
| Batch 022400_18 | 18: | PURSUIT ® | PURSUIT ® |
| Batch 022400_19 | 19: | PURSUIT ® | PURSUIT ® |
| Batch 022400_21 | 21: | PURSUIT ® | PURSUIT ® |
| Batch 022400_22 | 22: | PURSUIT ® | PURSUIT ® |
| Batch 022400_23 | 23: | PURSUIT ® | PURSUIT ® |
| Batch 030100_01 | 24: | Control | Control |
| Batch 030100_02 | 25: | Control | Control |
| Batch 030100_04 | 27: | Control | Control |
| Batch 030100_06 | 29: | Control | Control |
| Batch 030100_07 | 30: | PURSUIT ® | PURSUIT ® |
| Batch 030100_09 | 32: | PURSUIT ® | PURSUIT ® |
| Batch 030100_10 | 33: | PURSUIT ® | PURSUIT ® |
| Batch 030100_12 | 35: | PURSUIT ® | PURSUIT ® |
| Batch 030100_13 | 36: | PURSUIT ® | PURSUIT ® |
| Batch 030100_14 | 37: | PURSUIT ® | PURSUIT ® |
| Batch 030100_15 | 38: | PURSUIT ® | PURSUIT ® |
| Batch 030100_16 | 39: | PURSUIT ® | PURSUIT ® |
| Batch 030100_17 | 40: | PURSUIT ® | PURSUIT ® |
| Batch 030100_18 | 41: | PURSUIT ® | PURSUIT ® |
| Batch 030100_19 | 42: | PURSUIT ® | PURSUIT ® |
| Batch 030100_20 | 43: | PURSUIT ® | PURSUIT ® |
| Batch 030100_21 | 44: | PURSUIT ® | PURSUIT ® |
| Batch 030100_22 | 45: | PURSUIT ® | PURSUIT ® |
| Batch 030100_23 | 46: | PURSUIT ® | PURSUIT ® |
| Batch 030100_24 | 47: | PURSUIT ® | PURSUIT ® |
| Batch 030600_01 | 48: | Control | Control |
| Batch 030600_02 | 49: | Control | Control |
| Batch 030600_03 | 50: | Control | Control |
| Batch 030600_04 | 51: | Control | Control |
| Batch 030600_05 | 52: | Control | Control |
| Batch 030600_06 | 53: | Control | Control |
| Batch 030600_07 | 54: | Sethoxydim | Sethoxydim |
| Batch 030600_10 | 57: | Sethoxydim | Sethoxydim |
| Batch 030600_11 | 58: | Sethoxydim | Sethoxydim |
| Batch 030600_12 | 59: | Sethoxydim | Sethoxydim |
| Batch 030600_14 | 61: | Glyphosate | Glyphosate |
| Batch 030600_15 | 62: | Glyphosate | Glyphosate |
| Batch 030600_18 | 65: | Foul | Foul |
| Batch 030600_19 | 66: | Diuron | Diuron |
| Batch 030600_20 | 67: | Diuron | Diuron |
| Batch 030600_23 | 70: | Diuron | Diuron |
| Batch 030600_24 | 71: | Diuron | Diuron |

### Example 5

### Examples for Evaluation of the Limits of the NN Approach

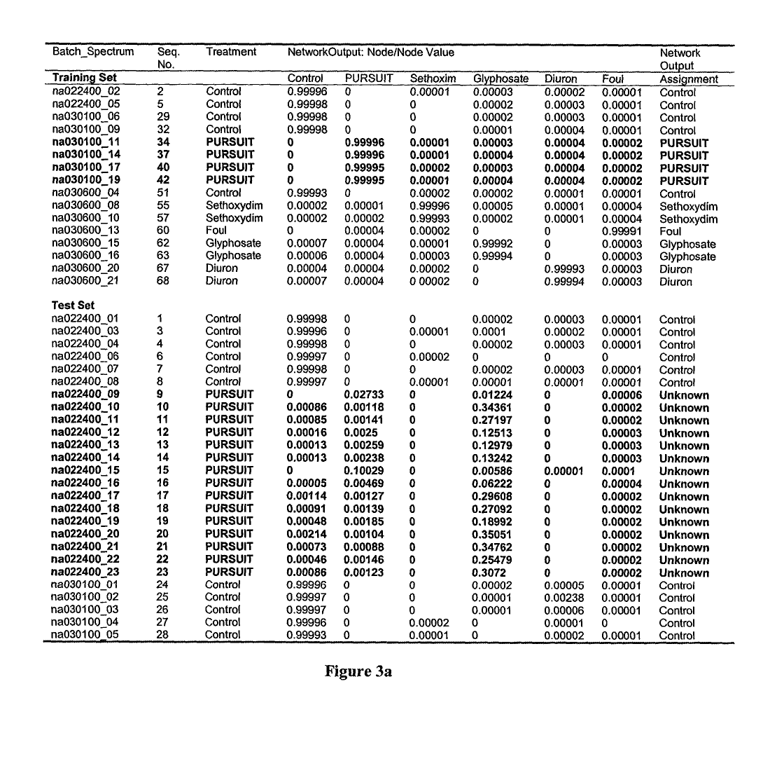

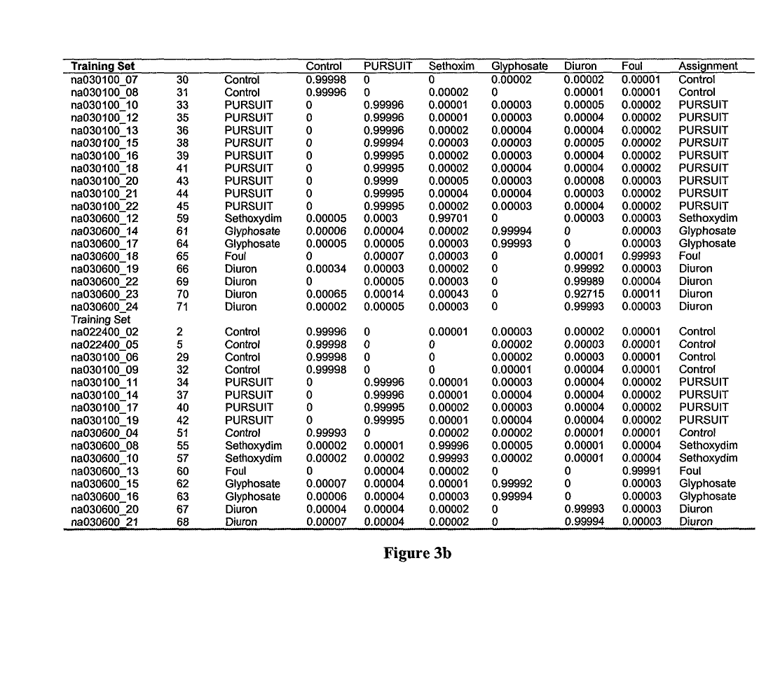

In an attempt to examine the limits of the approach, a variety of experiments were performed with distorted conditions to evaluate cases under which the network approach might fail: See FIGS. 3a and 3b.

Full recognition failed for the following cases:

If a treatment type was not represented in the training, recognition could not be achieved. Such samples were classified as unknown. Furthermore, stable recognition required at least 2 examples for each treatment.

Changes in experimental conditions, e.g. a temperature change of a few degrees in during the NMR spectral acquisition, yield samples as “Unknown”, unless the training set contains examples of the modified conditions. For example, as shown in FIG. 3a and 3b, spectra of one of the PURSUIT® treated batch were recorded at a 3° C. higher temperature. If no spectrum of this batch was presented to the network, a network trained with PURSUIT®-treated samples of the other batches failed to recognize all samples from the batch recorded at a higher temperature, and vice versa. From the output of that “designed-to-fail experiment” it also becomes apparent that, while usually spectra are recognized with activation values of >0.995 for each output node, the spectra of the PURSUIT® treated samples that were recorded at a higher temperature are having low activation values at the output node assigned to Glyphosate treated samples. This is due to the fact that some of the most significant resonance lines of the PURSUIT® treated samples are shifted upon temperature change to partially overlap with other resonance lines that are significant for detecting glyphosate treatment. However, by using not only those lines but a larger part of the spectrum with many other resonance lines, the NN still clearly distinguishes temperature-shifted PURSUIT® spectra from Glyphosate treated spectra by the low activation values. In general, activation below 0.6 is usually considered an indication for “not recognized”. Between 0.6 and 0.85 we can conclude that there is some similarity but no full identity of the treatment. Values larger than that indicate close proximity of treatments. Identical treatments for this data set have always resulted in output node activation values of >0.95, even if the training set was chosen to be a poor representative of the data space, like when only one or two representatives of each treatment were used. For a properly trained network within this example set, we always find activation values for the output nodes of >0.99 for recognition of a validation set treatment.

Training of the NN with sub-regions of the NMR spectra can yield recognition of treatments with sensitivity similar to using the full spectrum. However, the range of treatments that can be recognized is smaller. For example, using only the high-field portion of the NMR spectrum, that contains, among others, the resonance lines of aromatic protons, Controls, PURSUIT®-, Glyphosate- and Sethoxidym-treated samples could be fully recognized by properly trained NNs. However, training of Diuron treated samples with such trained networks appeared less specific, in particular if the amount of spectra in the training set is reduced to two or three per treatment In such cases we found occasionally false positive assignments. This result can be explained by analysis of the NMR-spectra: Diuron treated samples show most changes versus controls in the resonance region of the sugar-proton. Since this region was excluded in this particular experiment, Diuron treatment was only recognized if a larger amount of test spectra was used to highlight the very small changes between Diuron-treated and otherwise treated samples that are still present in the region of the aromatic protons. We thereby concluded that for general purposes, the use of the full spectral region is preferable. However, testing, evaluation, and specific detection systems may still use localized regions of the spectrum. Such approaches can, in some circumstances, reduce the time to train the network, or provide higher sensitivity for comparison of specific subsets of treatments. This observation also leads us to propose that a combination of NNs trained with different subsets of spectra or different regions of spectra or similar combinations can be use to produce several complementary NNs that can be used in combination to reach results for specific questions. The summary of results can then be presented to a “jury”, i.e., analyzed to reach a refined conclusion. Such approaches might become more important when larger numbers of treatments are being used in the experimentation, and a single network approach reaches a limit.

Training of a NN with only one or two treatment examples can produce other cases of false positive assignments. Such procedure leads to insensitive networks that, depending on the conditions and selection of training sets can frequently produce false positives or false negatives. This is due to a lack of generalization. We can conclude from such results that a larger number of samples for training may become necessary if samples variability (within one or more classes) increases, regardless of the difference between samples of different classes.

Detection of false negatives: Using only a small portion of the spectra (resonance region of aromatic protons) and training with very small sets of training spectra we produced networks that begin to loose their ability to perfectly recognize the samples. We had found earlier that the recognition was more stable if samples from different batches were used. In this case, using only a small portion of the NMR spectrum and only samples from Batch 1 to represent Control samples within the training set, we found that two individual samples of other batches of Controls were not automatically recognized. The activation values for those samples indicated that they would belong to either Controls (activation values for disputed samples were 0.990 and 0.980, respectively) or to Sethoxidym treated samples (respective activation values were 0.956 and 0.77). We conclude that a) the batches as a whole were clearly assigned to Controls; b) all other assignments were unaffected, c) as observed before, a representative training set and use of full spectral response can avoid such problems. It is noteworthy that performing the same experiment using spectra of Controls from either batch 2 or 3 in the training set, exclusively to represent Controls, does not produce a similar effect and all spectra are properly recognized, indicating that only batch 1 does not fully represent the variability within the Control spectra.

In almost all cases, as soon as some representative samples of each treatment group is present in the training set, recognition is perfect or nearly perfect. For a well-balanced training set, with little bias between individual batches, in many cases perfect recognition is achieved with two representatives for each treatment group. However, additional training set members, in particular when sampled from varying batches generally increases robustness. Part of the experimental variability can be simulated by adding noise too the spectra. For example, computer generated random values or noise spectra from the NMR instruments (using a sample with buffer only) can be added to the spectra of the training set to artificially increase the number of spectra for NN training. Similarly, shifting the spectra by one or two data points to the left or to the right can be applied to simulate effects of temperature changes in the NMR experiments. We found that small alterations improve robustness, while larger changes might reduce recognition.

We conclude that changes in the spectral response caused by changes in the treatments are large enough to allow robust distinction between treatments, while variability within similar treatments is small enough to require only a rather small amount of spectra for training. To produce a more widely applicable NN is preferable to include a larger, representative set of spectra in the training set and select example spectra that represent best the experimental diversity, e.g. different batches, slight variations in experimental conditions, etc.

### Example 6

### Generalization of the NN and Use of an NN for Recognizing the Mode-of-Action

The following example demonstrated that a NN that is trained with a one representative inhibitor of a pathway can recognize other inhibitors of that pathway even if the chemistry of these inhibitors is very different. As an example, we have used the NN trained and validated as shown in the above Example 3. It was trained to recognize untreated, PURSUIT®, Diuron, Sethoxydim, and Glyphosate treatment. In a blind test, we presented pattern from samples that were treated with no herbicide or different concentrations of various herbicides. In addition to the herbicides used in the training set, two other imidazolinones: ASSERT® and ARSENAL® (imazapyr and imazamethabenz) and two sulfonylureas GLEAN® and OUST® (chlorsulfuron and sulfometuron) were chosen, and the plants were treated as described above. For the blind test of the ANN analysis tool the samples only the two first to batches contributed samples to the training set. Thereby, the neuronal network had to truly recognized new batches with many samples having compound treatments applied that were unknown to the NN. In summary, we found a complete success of the methodology: The neuronal network classifies all untreated samples correctly as untreated, assigns the correct herbicide treatment for all herbicides that have been previously presented to the NN during training, even if such samples originated from batches that were not part of the NN training. Furthermore, the NN also classified with very high confidence all treatments with herbicides that are AHAS inhibitors, such as OUST®, ARSENAL®, etc., into the same class than PURSUIT®, even so the NN has never been trained with any AHAS inhibitor other than PURSUIT®, i.e. all herbicides had been correctly assigned as AHAS inhibitors, even so the herbicides used are of different chemistry.

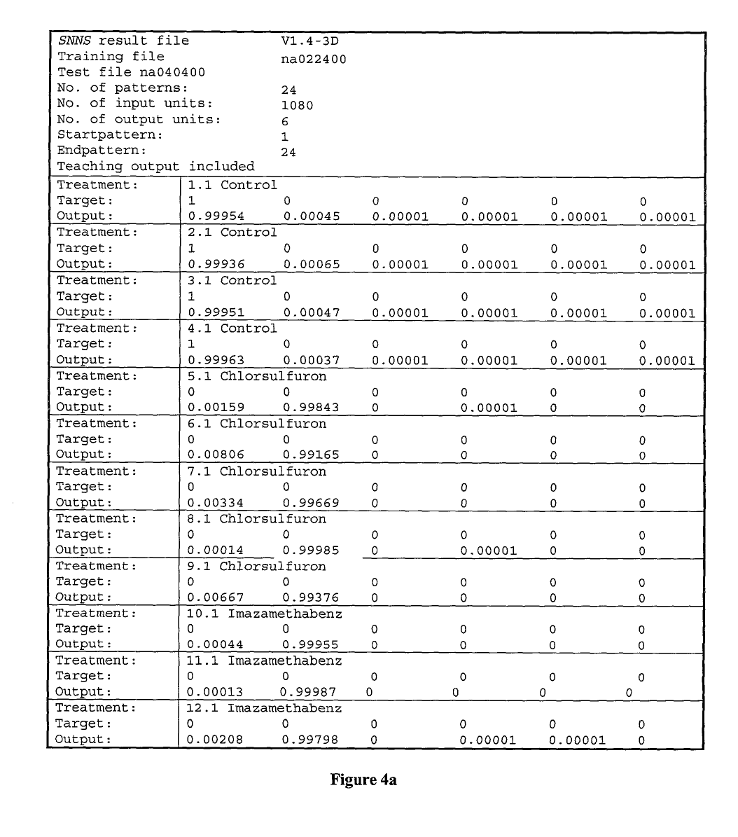

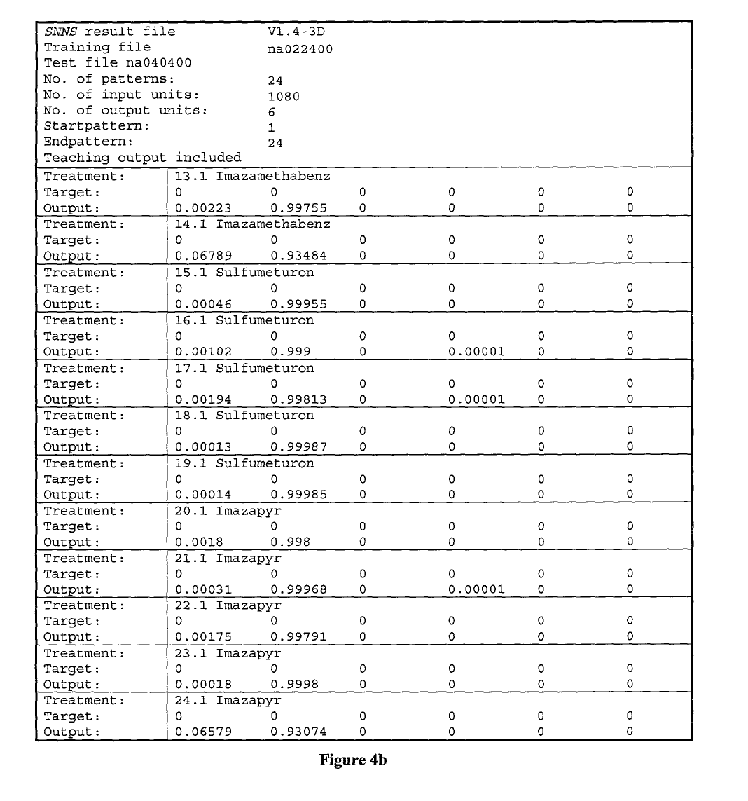

Note that the learning output target for the test spectra is zero in all cases in FIG. 4a and 4b. The total SSE in the calculation was high because of the difference between the given output value (zero) and the calculated value, but the spectra were correctly classified in all cases as belonging to the second output node, which is imazethapyr or AHAS inhibition. Similar results were obtained for the second set of experiments.

We conclude that selection of a comprehensive and well balanced training set with samples from separate batches representing the treatment cases will produce powerful NNs that can robustly recognize many different treatments even if the spectral changes are minute.

### Example 7

### Recognition of Gene and Genetic Alterations

As a prelude to determining the functional genomics applications of this methodology, we designed experiments to investigate whether the metabolic profiling method is capable of detecting differences in germ line as well as alterations in the metabolic profile caused by the effect of a genetic alteration.

In these experiments, seedlings from three genetically different corn seed lines were germinated, grown in hydroponic medium, excised, extracted and measured as described before. The plants belong to “wild type” (WT, Pioneer 3514, PURSUIT® sensitive), imidazolinone-tolerant (IT, Gerst 8541 heterozygotic, PURSUIT® tolerant), and imidazolinone-resistant (IR, Pioneer 3395, homozygotic, PURSUIT® resistant) lines.

Besides from light phenotypic variations between them, the difference between the three lines resides mainly on a mutation on the ahas gene. This mutation causes an asparagine to serine mutation in the AHAS protein at a specific position which leads to reduced inhibition of the mutated AHAS protein by imidazolinone herbicides than the wild type. IT lines are heterozygous for this mutation. IR lines are homozygous for this mutation.

The following experiments were designed to establish whether small genetic changes on a plant species can be detected by pattern recognition technology.

Two batches with five seedlings each from WT, IR, and IT lines, were grown at various levels of PURSUIT® concentration, as follows in Table 5:

TABLE 5
Number of samples for each corn line grown under various
PURSUIT ® concentrations. Numbers are given for the first and
second batch of each line and PURSUIT ® concentration

| [PURSUIT ®] | WT corn | IT corn | IR corn |
|---|---|---|---|
| 0.0 mM (control) | 5/5 | 5/5 | 5/5 |
| 0.041 mM | 5/5 | 5/5 | 5/5 |
| 0.166 mM | 5/5 | 5/5 | 0/5 |
| 0.666 mM | 5/5 | 5/5 | 5/5 |
| 2.65 mM | 5/5 | 5/5 | 5/5 |
| Saturated sol. (>4 mM) | 515 | 5/5 | 5/5 |

The seeds used for these experiments derive from different lines and even from different seed companies. During germination, growth and harvest of the seedlings it was observed that the phenotypes were slightly different, besides of the herbicide tolerance. Some of the seeds, in particular IT and IR showed a lower germination rate. Also the leaves of IT are shorter and wider than the leaves from WT plants. Furthermore, it was observed that the seedlings from IT and IR lines had a more heterogeneous pattern of growth: some of the IT and IR lines did not reach the three leaf stage by the end of the fifth day, as was consistently observed in the WT seedlings.

Some of the plants used for the experiments were in an earlier stage of development in the first batch of seedlings. Most of the younger plants were taken for the controls. In the second batch, more seeds were put to germinate so that enough plants should have reached a stage mature enough to submit them to the treatment.

The different lines of corn can be distinguished phenotypically at growing levels of herbicide treatment. The phenotypic response observed is a total arrest on the growth of the plants and their wilting within 48 hours. For WT, herbicidal effects are already observed at the lowest (41 μM) PURSUIT® concentration. On the other hand even the IR lines are affected by concentrations of imidazolinones so high as 4 mM. The plants were harvested after only 24 hours after treatment such that phenotypic differences were restricted to the development (or lack) of the fourth leaf. It is important to harvest at an early stage to avoid that the plants become senescent. The senescence process produces accumulation of a series of metabolites that would obscure the metabolic profile response associated with one specific mode of action.

In a first NN training and validation, the two batches from each WT, IT, IR line grown without herbicide (control plants) were used. The metabolic profile analysis was performed essentially as described above. It was found that it is difficult to distinguish the pattern for WT, IT and IR lines. Statistical analysis of data variability indicates that WT, IR, and IT spectra are different but intra- and inter-batch variability is almost of the same order of magnitude. In particular for the first batch, where plant material was less well selected for similar development stage due to the limited number of available seedlings at those days, recognition of all types is found to be somewhat dependent on the choice of data sets that are used for network training.

We found that samples from one batch alone, or a selection of 1-2 samples from each batch are not sufficient to generate a reliable NN. The choice of the samples for training and even some of the parameters from the training partially affect the outcome of the validation runs. However, if many samples (2-3) for each seed group (WT, IT, IR) from both batches are used for training the network, the remaining samples are classified correctly with typically 1 or 2 samples being classified as unknown. However, this does not affect the overall result, and in all cases, the batch as a whole can be classified correctly.

In Table 6, the first data row indicates that for class 0 (WT) there is no sample classified correctly, one sample classified wrongly as class 1 (IT) and 4 samples classified as unknown, probably reflecting the difference in the developmental stage of these four plants. The other values in Table 8 show that IT and IR lines are also confused and a majority of the samples cannot be classified correctly. We can conclude that, under these conditions, variations between different batches are obscuring possible genetic variability.

If the network is trained with 6 samples (2 samples from each plant type) from each batch, i.e., a total of only 12 samples used for training, and validated using the remaining 18 samples, the network is capable of tolerating the variation in the developmental stage and between the batches. The validation results shown in Table 7 indicate that the majority of the samples from the validation batches are correctly recognized. The network error that is reported in the header of each table is the sum of the quadratic differences between the teaching input and the real output over all output.

TABLE 6
Summaries of the results of network validation from a network
trained with all 15 samples of batch 2, and validated with 15 samples
from batch 1. The results are displayed in form of a “confusion
matrix”, with rows representing the correct answer, and columns the
result from the network prediction. The network error for this
validation is 14.5.

| Class | WT | IT | IR | Unknown |
|---|---|---|---|---|
| WT | 0 | 1 | 0 | 4 |
| IT | 2 | 3 | 0 | 0 |
| IR | 3 | 1 | 1 | 0 |
| No class | 0 | 0 | 0 | 0 |

TABLE 7
Summaries of the results of network validation for a NN trained
with only 6 samples (2 samples from each plant type) from each batch
and validated using the remaining 18 samples, displayed as in Table
8. The NN error for this validation set is 4.40

| Class | WT | IT | IR | Unknown |
|---|---|---|---|---|
| WT | 5 | 1 | 0 | 0 |
| IT | 0 | 4 | 0 | 2 |
| IR | 0 | 0 | 4 | 2 |
| No class | 0 | 0 | 0 | 0 |

In the following analysis, we evaluate whether an addition of PURSUIT® as an AHAS inhibitor leads to a more pronounced distinction between the lines, which would indicate that the alteration in residual AHAS activity due to the herbicide-resistance mutation in the IT and IR lines are affecting the overall metabolic pattern in a distinctive way that can be detected by the pattern analysis.

Using the exact same setup of the experiment as before, but applying 66 mM PURSUIT® into the growth media, the distinction between the lines is more pronounced. A wide variety of NNs, generated with different sample selections for training the network, all yield very satisfactory results. Only a few samples chosen from each batch for training the network are sufficient to create robust NNs that classify the batches with high confidence.

TABLE 8
Validation results from a network trained with 12
samples (2 samples from each batch, 2 batches of each line).
All validation samples have been correctly recognized with
a network error of 0.17.

| Class | WT | IT | IR | Unknown |
|---|---|---|---|---|
| WT | 6 | 0 | 0 | 0 |
| IT | 0 | 6 | 0 | 0 |
| IR | 0 | 0 | 6 | 0 |
| No class | 0 | 0 | 0 | 0 |

In the third part of this experiment, we analyze recognition of the metabolic profile for samples that are treated at with a saturated solution of PURSUIT®. Under these conditions, even IR plants are known to show growth arrest.

### Example 8

### Simultaneous Analysis of Herbicide Mode-of-Action Recognition

The present example describes the simultaneous analysis of nineteen MOAs in a single, very large neural network developed from 299 NMR spectra of plant isolates. Corn plants (Zea mays) were treated with various herbicides such as imazethapyr, glyphosate, sethoxydim, and diuron, which represent various biochemical modes-of-action such as inhibition of specific enzymes (acetohydroxy acid synthase enzyme [AHAS], protoporphyrin IX oxidase [PROTOX], enzyme 5-enolpyruvylshikimate-3-phosphate synthase, [EPSPS], acetyl CoA carboxylase [ACC-ase], etc.), or protein complexes (photosystems I and II), or major biological process such as oxidative phosphorylation, auxin transport, microtubule growth, and mitosis. Crude isolates from the treated plants were subjected to ¹H NMR spectroscopy, and the spectra were classified by artificial neural network analysis to discriminate the herbicide modes-of-action. Of the nineteen MOAs studied in a single large neural network, the control group (untreated), AHAS, ACCase, EPSPS, PROTOX, carotenoid, PSI, uncoupler, auxin-like, auxin transport, acetamide-like, PSII, and glutamine synthase inhibitors were all well classified, whereas HPPD, PDS, DHP, microtubule, and mitosis inhibitors were not well classified. A larger sample population may be needed to classify these MOAs. Taken together, the PSI_c1 and PSII_c2 photosynthesis II subclasses were classified correctly as PSII inhibition in most of the treated plants, but these subclasses were strongly confused with each other. In contrast, subclass PSII_c3 was always readily distinguishable from the other PSII subclasses.

Plant Growth Conditions

Zea mays seeds (Pioneer 3514) were set to germinate in paper towel rolls in tap water for 5 days in the growing chamber. The environment was adjusted to “summer conditions” (day/night ratio of 14/10 hours, regulated temperature of 27° C. and humidity of 70%). After germination the seedlings were visually inspected. Seedlings that were homogeneous in size and appearance were selected, set in 50-ml amber bottles in 25-ml Hoagland nutrient solution (12 ml micronutrients stock solution, 12 ml FeEDTA (5 g/100 ml), 2.4 ml KH₂PO₄ (1 M), 24 ml MgSO₄ (1 M), 60 ml KNO₃ (1 M), 60 ml Ca(NO₃)₂ (1 M), and 60 ml MES buffer (200 mM), diluted to 12 litre with deionized water) and grown for 5 more days, after which they reached the three-leaf stage. At this point, 20 μl of a stock solution of technical grade herbicide in acetone (see Table I) was added to the hydroponic solution or applied to the second leaf (with similar results). The control group of “Untreated Plants” received 20 μl acetone only and all of the plants were returned to the growing chamber.

### Extraction and Sample Preparation

Twenty-four hours post-treatment, the plants were harvested by excising between the coleoptile and the first leaf collar. At this time, the plants show only slight growth stunting in response to the treatments. The first leaf sheet was separated and the meristematic tissue (approximately 250 to 300 mg per plant) was collected, flash frozen in liquid nitrogen in a cryogenic 3 ml tube, and stored in a liquid nitrogen freezer until further use. The plant meristems were each pulverized in a mortar (under liquid N₂), suspended in 2.4 ml of HCl solution (0.25N) and centrifuged at 14000 g, 4° C., for 60 minutes. The NMR samples were prepared from 0.8 ml of the supernatants and 0.2 ml D₂O (with TSP 0.05 w/v) and kept on ice.

### Treatment Herbicides

TABLE 9
Herbicides Used in the NMR Metabonomics Experiments

| Herbicide | Structure | Herbicide | Structure |
|---|---|---|---|
| Imazethapyr | 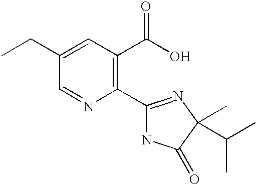 | Sulfometuron | 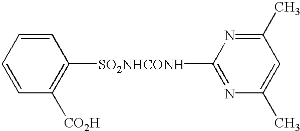 |
| Imazametha- benz m- and p- isomers | 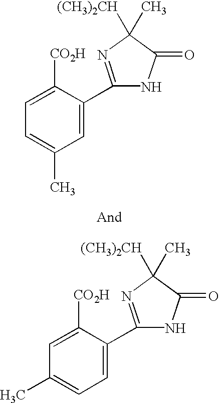 | Diuron | 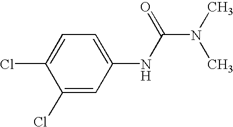 |
| Imazapyr | 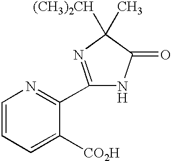 | Sethoxydim | 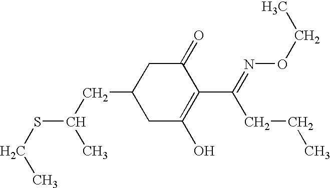 |
| Glyphosate | 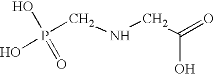 | Chlorsulfuron | 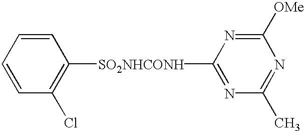 |
| Bialaphos* (Bilanafos) | 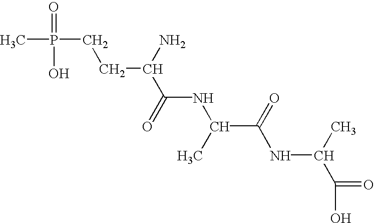 | Glufosinate | 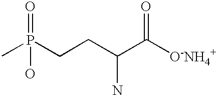 |
| Lenacil | 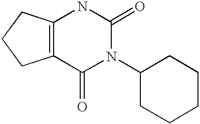 | Asulam | 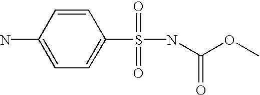 |
| Bromoxynil | 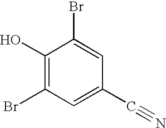 | Oryzalin | 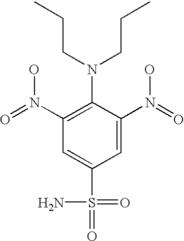 |
| Paraquat | 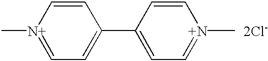 | Chlorpropham | 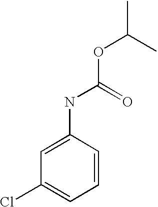 |
| Acifluorfen | 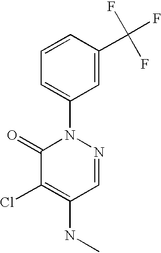 | Propham | 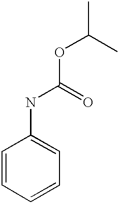 |
| Norflurazon | 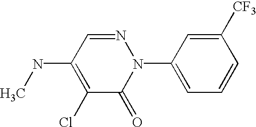 | Carbetamide | 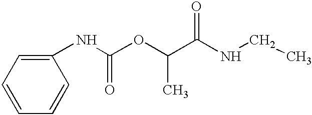 |
| Sulcotrione | 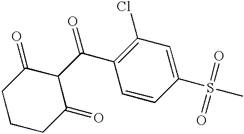 | Acetochlor | 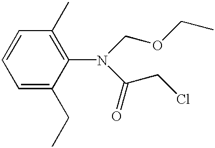 |
| CMPD 1 | 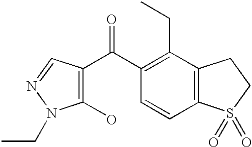 | Dichlobenil | 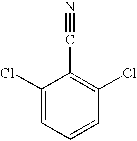 |
| CMPD 2 | 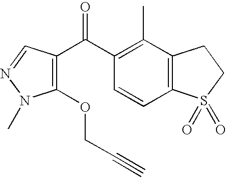 | Chlorthiamid | 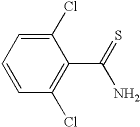 |
| CMPD 3 | 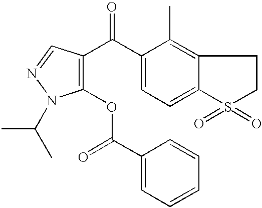 | Dinoseb | 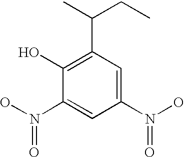 |
| CMPD 4 | 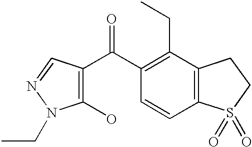 | Quinclorac | 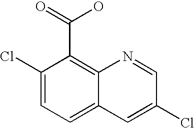 |
| Amitrole | 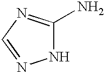 | Naptalam | 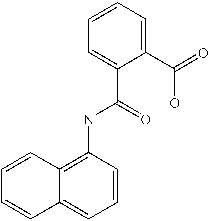 |
| CMPD 5 | 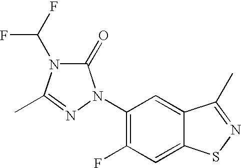 | | |

Zea mays plants were treated post-emergence with the herbicides shown in Table 9.

NMR Spectroscopy

NMR Acquisition

The 500 MHz ¹H NMR spectra of plant extracts were recorded using a Bruker AMX 500 NMR spectrometer equipped with a TXI 5 mm probe. The probe temperature was carefully regulated using the Bruker/Haake variable temperature accessory, and all spectra were recorded under identical experimental conditions, as shown in Table 10:

TABLE 10
Standardized NMR Acquisition Parameters

| Parameter | Setting |
|---|---|
| Pulse program: | zgpr (solvent presaturation at O1) |
| Time domain: | 16384 points (complex points) |
| Number of scans: | 256 |
| Number of dummy scans: | 256 (10 min for temperature equilibration) |
| Temperature: | 295K (22° C.) |
| Spectral width: | 5555.56 Hz |
| Acquisition time: | 1.47461 sec |
| Receiver gain: | 256 |
| Dwell time: | 128.57 |
| HL1 power: | 3 dB |
| D12 delay: | 20 μsec |
| HL2 power: | 60 dB (for water presaturation) |
| P18 (water sat. pulse): | 1 sec |
| D13: | 4 μsec |
| P1: | 4 μsec (transmitter high power pulse) |
| SFO1: | 500.1323559 MHz (transmitter frequency) |

NMR Processing

The time-domain NMR spectra (“FIDs”) were exponential multiplied (LB=0.5 Hz), Fourier transformed, and then phase- and baseline-corrected manually. The frequency domain were exported by the NMR software as J-CAMP formatted files, which were stored in a UNIX subdirectory for “preprocessing” via NN_Tools, as described below.

Preprocessing by NN_Tools

The frequency domain NMR spectra were “preprocessed” by NN_Tools as follows: J-CAMP formatted spectra files were converted into vectors (8 k real data points), and the files were renamed (renumbered) in order to be processed by further programs in an automatic fashion. A window of points was cut from the central part (around O1) of each vector to delete the residual water signal. Then points were cut from the low field and high field parts of the vector, because no resonance signals were detectable in these regions. Groups of typically five (5) adjacent points were averaged in histogram fashion (“bining”) and the resulting “preprocessed” spectrum comprised 1080 data points. Finally, vertical scaling was applied to meet the signal amplitude requirements of the neural network software.

Neural Network Computation

The artificial neural network calculations described in the report were performed using a standard software package, the Stuttgart Neural Network Simulator (SNNS), on a Silicon Graphics Inc. (SGI) UNIX workstation. A convenient interface called NN_Tools, was developed in-house to perform NMR spectral preprocessing and to format the raw data for input to SNNS. NN_Tools comprises a set of Perl scripts which form patterns out of NMR spectra that can be input automatically to SNNS for the training, validation, and testing steps of neural network simulation. This free software package (“freeware”) was developed at the Institute for Parallel and Distributed High Performance Systems at the University of Stuttgart, Germany. SNNS Group, Institute for Parallel and Distributed High-Performance Systems (IPVR), University of Stuttgart, Breitwiesenstrasse 20-22, 70565 Stuttgart, Fed. Rep. of Germany, Zell, A. (2000) Simulation neuronaler Netze, R. Oldenbourg Verlag, München). The function that produced the most reliable, reproducible results with the lowest error in recognition was “resilient back-propagation” (coded in the Rprop module of SNNS), which is a local adaptive scheme performing supervised training in a multilayered network, as described above.

The learning parameters for this example are shown in Table 11.

TABLE 11
Optimal Learning Parameters for SNNS

| Parameter | Value |
|---|---|
| Learning function: | Resilient Back Propagation |
| Update function: | Topological Order |
| Initialization Function: | Randomize Weights |
| Initial update value: | 0.1 |
| Maximum step size: | 50 |
| Number of layers: | 3 (1 Input, 1 Hidden, 1 Output) |
| Input layer: | 1080 Nodes |
| Hidden layer: | 12 Nodes |
| Output layer: | 6 Nodes |

These parameters were used for all calculations described in the following section. The test sets were presented every 10 or 20 steps of training, and the training was done in cycles of 25 steps, after which the network status was saved and the error file printed on the screen and into a file. This procedure was repeated for 20 epochs (500 cycles total) and the best net was chosen by a script that identifies the state with smallest residual error. This process effectively avoids overtraining.

Neural Network Analysis for Nineteen MOAs

A neural network calculation was performed using the NMR spectra of 299 plant isolates as input. These isolates represent nineteen (19) different herbicide modes-of-action. The calculation was performed in two different ways:

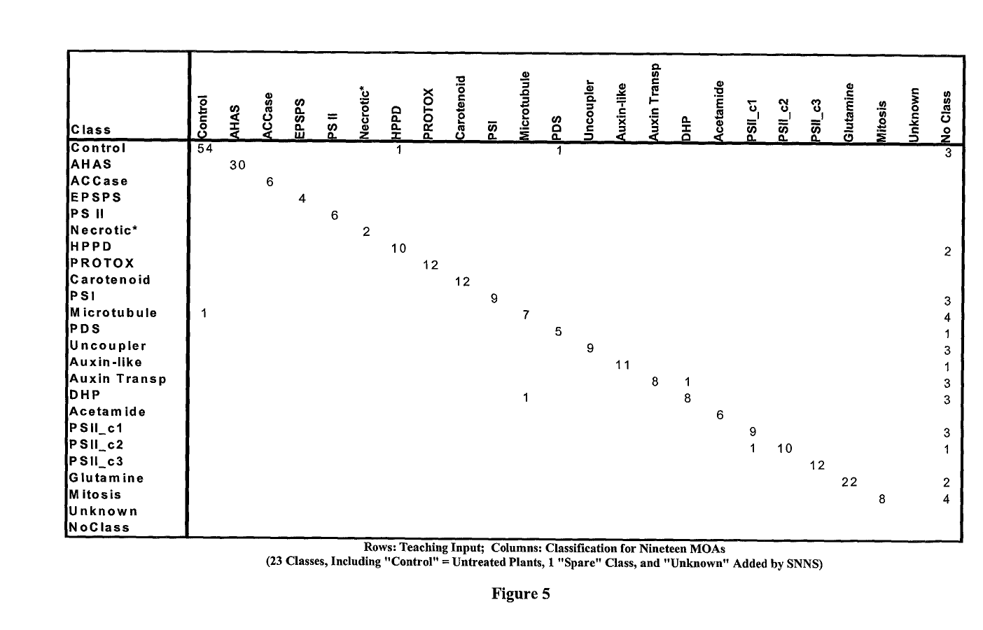

1. In the first calculation (“Calculation A”), a random sampling of 145 spectra was used for training and the full set of 299 spectra was used for testing. FIG. 5 (Notes: The matrix shows the total number of plants classified by the neural network according to the classes given as teaching input. For example, of the 59 control (untreated) plants, 54 were correctly classified, 2 plants were confused with HPPD and PDS treatments, and 3 plants were unrecognized. The “necrotic” class includes two glyphosate-treated plants that were obviously senescent and showing signs of decay and whose NMR spectra differed greatly from other .glyphosate-treated plants.) shows the so called “Confusion Matrix” that is also generated by the SNNS software. For example, of the 59 control (untreated) plants, 54 were correctly classified, 2 plants were confused with HPPD and PDS treatments, and 3 plants were unrecognized. The “necrotic” class includes two glyphosate-treated plants that were obviously senescent and showing signs of decay, and whose NMR spectra differed greatly from other glyphosate-treated plants.

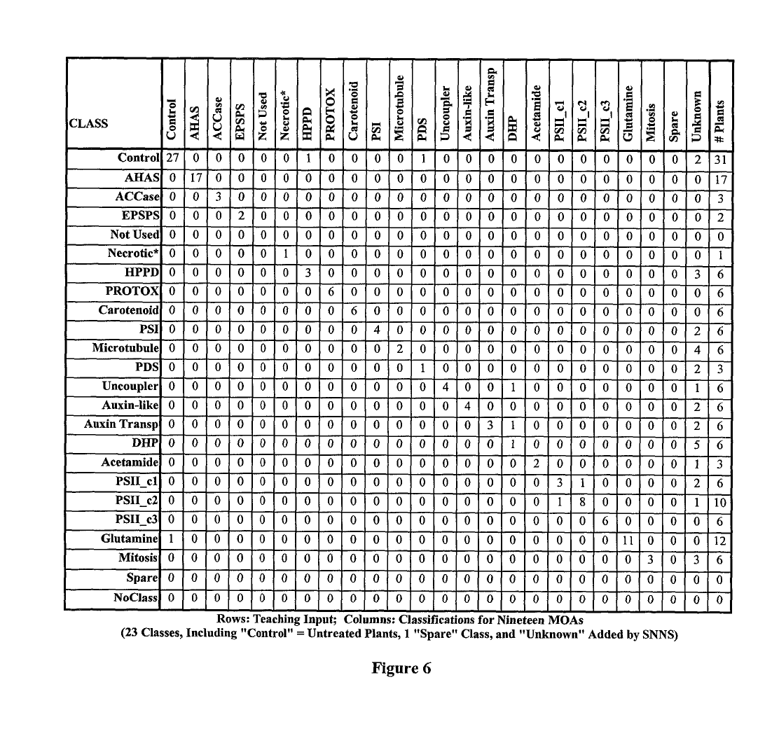

2. In the second calculation (“Calculation B”), the same random sampling of 145 spectra was used for training and the remaining 154 spectra (299−145=154) were used for testing. Thus, the training and testing sets are fully independent. FIG. 6 (Notes: The matrix shows the total number of plants classified by the neural network according to the classes given as teaching input. For example, of the 31 control (untreated) plants, 27 were correctly classified, 1 plant was confused with HPPD treatment, and 3 plants were classified “unknown”. The “necrotic” class includes two glyphosate-treated plants that were obviously senescent and showing signs of decay and whose NMR spectra differed greatly from other .glyphosate-treated plants.) shows the corresponding “Confusion Matrix” as generated by the SNNS software.

### DISCUSSION

Growing Conditions

One of the most important requisites for the work on metabolic profiling in plants is the reproducibility and stability of the physical conditions in which the plants are grown. Plants, as all living organisms, react to different environmental stimuli and changes turning on and off different genes, expressing different proteins and enzymes, and developing different metabolic states, usually the most appropriate for the best development of the organism in the given environment.

In the early developmental stage (5 to 10 days after germination) in which the seedlings in this study were treated and harvested, metabolic changes are fast and changes in the concentrations of metabolites are considerable for the small amount of growing point tissue that can be collected. Relative small changes in the environment of a plant can be reflected in very detectable variations in the absolute concentration of a metabolite and with that, a change of the profile.

For these reasons, the use of growing chambers, where the environmental conditions can be accurately controlled, is preferred. In the course of the present study, for example, some plants had to be transferred from one growing chamber to another, due to the mechanical failure of the first one. Some hours of more elevated temperature and then change in the illumination, produced in the plants metabolic profiles that were classified by the ANN as an unknown species.

NMR Spectroscopy

The use of an acidic matrix to prepare the extracts of plant tissue allowed us to get the widest range of primary metabolites (amino acids, sugar, sugar-alcohols, organic acids, etc.). Due to the relative low sensitivity of NMR spectroscopy, it is important to choose as many of the metabolites present in the highest concentrations as probes for the total metabolic profile. Another reason to choose this extraction matrix is that it does not produce any undesirable solvent peaks in the NMR spectrum. The steps and procedure for the extraction were optimized to give the highest possible throughput without losing sensitivity in the analysis response.

Reproducibility of conditions is the key for a reliable classification of the spectra. Temperature and spectral width seem to be the most important factors. The exact total concentration of metabolites in the sample (which is dependent on the amount of tissue used for extraction) is less critical for two reasons: a) Use of an internal reference standard in each sample, and b) Normalization of all the spectral intensities as part of the processing of the spectra when preparing patterns for analysis with the ANN.

Although 8 K (8192) real points were used when acquiring the spectra, only 1080 points were needed for each pattern to be accurately recognized. The 500 MHz NMR spectrometer gives a very good resolution and signal to noise ratio. After 256 transients, more than 300 peaks can be automatically picked from the spectrum, which present a signal to noise ratio >30. Even the narrowest peaks are described by 10 data points or more. Different reductions of the number of spectral points were investigated by averaging a number of adjacent points into bins. Averaging each block of 5 contiguous points in the pattern to one point yielded very good results on the ANN analysis. This accelerates the computation considerably without loss of fidelity, a great advantage since many training methods and parameters had to be tested, and because the calculation of many spectra requires considerable time and hardware resources.

Special care was made to always use the same power level and pulse duration to irradiate the water signal, as differences in this factor may produce artifacts in the downfield part of the spectrum, especially in exchanging NH groups. As well, the residual water signal was completely cut from the spectrum (always between the same two spectral points) prior to NN analysis.

Many replicates of each sample were prepared and measured in each experiment. Usually five-to-twelve plants were grown, treated and harvested for each treatment class. Due to normal variation between individual organisms, this procedure is recommendable when constructing a database and when trying new modes-of-action. Each experiment was repeated at least twice at different times.

MOA Discrimination

In all, nineteen (19) different modes of action have been studied in Pioneer 3514 corn and most were successfully distinguished by the NMR metabonomics method. The results obtained to date are summarized in Table 12. The degree of discrimination among the various modes of action depend to a degree on how the data are analyzed. For example, the data can be processed in small groups of several MOAs. The results show for four herbicide treatment groups (imazethapyr, sethoxydim, glyphosphate and diuron) and a control group illustrate the virtually perfect discrimination among several herbicides with different modes-of-action. The relatively small neural network used was trained with spectra of a first batch of plants that contained the same treatment regimes as that of a second batch. The output unit activation is almost 1 in all cases, with no confusion among the MOAs.

A comparison of output unit activation vs. herbicide treatment group for several chemically different AHAS inhibitors (chlorsulfuron, imazamethabenz, sulfometuron, and imazapyr) was performed. The results demonstrate that all of these herbicides are classified by the neural network as “imazethapyr”, consistent with their mutual mode-of-action of AHAS inhibition.

TABLE 12
Summary of the Herbicides Examined by the Metabolic Profiling
Method for which the Modes-of-Action were Tested

| HRAC | | |
|---|---|---|
| Group | Mode-of-Action | Compounds |
| A | Inhibition of acetyl CoA carboxylase | Sethoxydim |
| | (ACCase) | |
| B | Inhibition of acetohydroxyacid synthase | Chlorsulfuron |
| | (AHAS, ALS) | Sulfometuron |
| | | Imazamethabenz |
| | | Imazapyr |
| | | Imazethapyr |
| C1 | Inhibition of photosynthesis at photosystem | Lenacil |
| | II | |
| C2 | Inhibition of photosynthesis at photosystem | Diuron |
| | II | |
| C3 | Inhibition of photosynthesis at photosystem | Bromoxynil |
| | II | |
| D | Inhibition of photosynthesis at photosystem I | Paraquat |
| E | Inhibition of protoporphyrinogen oxidase | Acifluorfen |
| | (PPO, PROTOX) | |
| F1 | Bleaching inhibition at phytoene desaturase | Norflurazon |
| | (PDS) | |
| F2 | Bleaching inhibition of 4-hydroxyphenyl- | Sulcotrione |
| | pyruvate-dioxygenase (HPPD) | |
| F3 | Carotenoid biosynthesis inhibition (unknown | Amitrole |
| | target) | |
| G | Inhibition of EPSP synthase | Glyphosate |
| H | Inhibition of glutamine synthase | Bialaphos* |
| | | Glufosinate |
| I | Inhibition of DHP (dihydropteroate synthase) | Asulam |
| K1 | Inhibition of microtubule assembly | Oryzalin |
| K2 | Inhibition of mitosis/microtubule | Chlorpropham |
| | organization | Propham |
| | | Carbetamide |
| K3 | Acetamide herbicide-like | Acetochlor |
| L | Inhibition of cell wall (cellulose) synthesis | Dichlobenil |
| | | Chlorthiamid |
| M | Uncouplers of oxidative phosphorylation | Dinoseb |
| O | Auxin-like (action like indole acetic acid) | Quinclorac |
| P | Inhibition of auxin transport | Naptalam |
| *Glufosinate and bialaphos are reported to have the same mode of action (inhibition of glutamine synthase). However, the NN analysis is not able to classify them into one bin. Unfortunately, the bialaphos used for this experiment was a formulation, while the glufosinate sample was a technical material. After 24-hours post-application, the plants that had been treated with bialaphos formulation | | |
| # presented much stronger signs of damage than all the others. Formulations usually produce an effect of faster absorption and sometimes translocation that increases the metabolic response. | | |

The discrimination among MOAs is not quite as good when data for all nineteen MOAs are analyzed in single, very large neural network. Nevertheless, these preliminary results are very supportive of the value of the method.

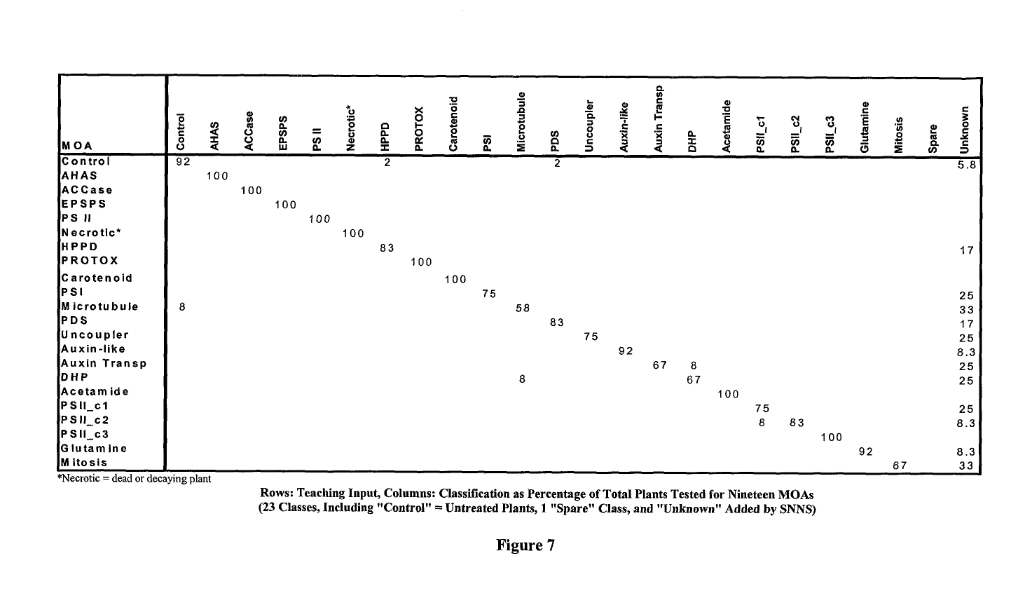

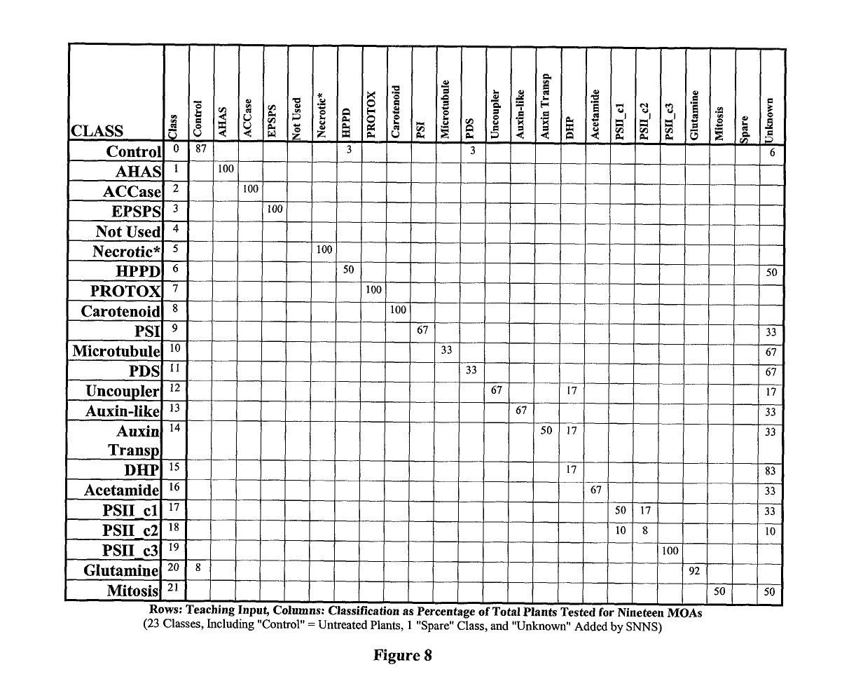

For “Calculation A”, utilizing 145 training spectra and 299 test spectra representing 19 Herbicide MOAs, the degree of confusion between actual and deduced classifications is shown in the “raw” confusion matrix in FIG. 5. The raw data in FIG. 5 also can be expressed as the percentage of correct classifications for each class, as shown in FIG. 7. The greatest degree of confusion was observed for “microtubule assembly inhibition” and “glutamate synthase inhibition” which were simply not recognized in many spectra (i.e. classified as unknown). Otherwise, the degree of confusion for each class is quite small.

These same 299 spectra were analyzed somewhat differently in “Calculation B”, where 145 randomly-selected spectra were used in the training step and the balance of 154 spectra were applied for testing. Thus, the training and testing sets are statistically independent. The confusion matrix is tabulated in FIG. 18. The greatest degree of confusion occurs for microtubule inhibition, auxin transport inhibition, DHP inhibition, and mitosis inhibition. Perhaps not surprising, PSII_c1 and PSII_c2 are confused primarily with each other, whereas PS_c3 is distinguished. Overall, more spectra are classified as “unknown” in this calculation, yet fourteen of the nineteen MOAs are correctly classified.

In conclusion, this work has shown the feasibility of ¹H NMR spectroscopy of plant extracts, in combination with artificial neural network analysis, to discriminate the modes-of-action of many different herbicides. Of the nineteen MOAs studied in a single large neural network, the control group (untreated), AHAS, ACCase, EPSPS, PROTOX, carotenoid, PSI, uncoupler, auxin-like, auxin transport, acetamide-like, PSII, and glutamine synthase inhibitors were all well classified, whereas HPPD, PDS, DHP, microtubule, and mitosis inhibitors were not well classified. A larger sample population may be needed to classify these MOAs. Taken together, the PSII_c1 and PSII_c2 MOAs were classified correctly as PSII inhibition in 81% of the treated plants, but these subclasses were strongly confused with each other. In contrast, PSII_c3 was always readily distinguishable from the other PSII subclasses. The method is reliable when the experimental conditions are well controlled and accurately kept under standard conditions. The software and interface used for data analysis allow one to construct a large, easily accessible database, and to add new data when new leads are investigated.

### APPENDIX I

### Classification of Herbicides According to Mode-of-Action

Herbicides are classified alphabetically according to their target sites, modes of action (MOA), similarity of induced symptoms, or chemical classes. The system was developed cooperatively between the Herbicide Resistance Action Committee (HRAC) and the Weed Science Society of America (WSSA) (see Schmidt, R. R.: HRAC Classification of Herbicides according to Mode-of-Action, Brighton Crop Protection Conference, in Weeds 1133-1140, 1997).

If different herbicide groups share the same mode or site of action, only one letter is used. In the case of photosynthesis inhibitors, subclasses C₁, C₂ and C₃ indicate different binding behavior at the binding protein D₁ or different classes. Bleaching can be caused by different ways and three subgroups, F₁, F₂ and F₃, are used. Growth inhibition can be induced by herbicides from subgroups K₁, K₂ and K₃. Herbicides with unknown modes or sites of action are classified in group Z as “unknown” until they can be grouped exactly. In order to avoid confusion with I and O, categories J and Q are omitted. New herbicides will be classified by HRAC/WSSA in the appropriate groups or, if the mechanism is new, in new groups (R, S, T . . . ).

TABLE 13
HRAC & WSSA Herbicide MOA Classification Codes

| HRAC | WSSA | | | |
|---|---|---|---|---|
| Group | Group | Mode-of-Action | Chemical Family | Active ingredient |
| A | 1 | Inhibition of acetyl CoA | Aryloxyphenoxypropionates | Clodinafop-propargyl |
| | | carboxylase | ‘FOPs’ | Cyhalofop-butyl |
| | | (ACCase) | | Diclofop-methyl |
| | | | | Fenoxaprop-P-ethyl |
| | | | | Fluazifop-P-butyl |
| | | | | Haloxyfop-R-methyl |
| | | | | Propaquizafop |
| | | | | Quizalofop-P-ethyl |
| | | | Cyclohexanediones | Alloxydim |
| | | | ‘DIMs’ | Butroxydim |
| | | | | (clefoxydim proposed) |
| | | | | Clethodim |
| | | | | Cycloxydim |
| | | | | Sethoxydim |
| | | | | Tepraloxydin |
| | | | | Tralkoxydim |
| B | 2 | Inbibition of acetolactate | Sulfonylureas | Amidosulfuron |
| | | synthase (ALS) | | Azimsulfuron |
| | | or | | Bensulfuron-methyl |
| | | Acetohydroxyacid synthase | | Chlorimuron-ethyl |
| | | (AHAS) | | Chlorsulfuron |
| | | | | Cinosulfuron |
| | | | | Cyclosulfamuron |
| | | | | Ethametsulfuron-methyl |
| | | | | Ethoxysulfuron |
| | | | | Flazasulfuron |
| | | | | Flupyrsulfuron-methyl-Na |
| | | | | Foramsulfuron |
| | | | | Halosulfuron-methyl |
| | | | | Imazosulfuron |
| | | | | Iodosulfuron |
| | | | | Metsulfuron-methyl |
| | | | | Nicosulfuron |
| | | | | Oxasulfuron |
| | | | | Primisulfuron-methyl |
| | | | | Prosulfuron |
| | | | | Pyrazosulfuron-ethyl |
| | | | | Rimsulfuron |
| | | | | Sulfometuron |
| | | | | Sulfometuron-methyl |
| | | | | Sulfosulfuron |
| | | | | Thifensulfuron-methyl |
| | | | | Triasulfuron |
| | | | | Tribenuron-methyl |
| | | | | Trifloxysulfuron |
| | | | | Triflusulfuron-methyl |
| | | | | Tritosulfuron |
| | | | Imidazolinones | Imazapic |
| | | | | Imazamethabenz |
| | | | | Imazamox |
| | | | | Imazapyr |
| | | | | Imazaquin |
| | | | | Imazethapyr |
| | | | Triazolopyrimidines | Cloransulam-methyl |
| | | | | Diclosulam |
| | | | | Florasulam |
| | | | | Flumetsulam |
| | | | | Metosulam |
| | | | Pyrimidinyl(thio)benzoates | Bispyribac-na |
| | | | | Pyribenzoxim |
| | | | | Pyriftalid |
| | | | | Pyrithiobac-na |
| | | | | Pyriminobac-methyl |
| | | | Sulfonylaminocarbonyl- | Flucarbazone-Na |
| | | | Triazolinones | Procarbazone-Na |
| C1 | 5 | Inhibition of photosynthesis | Triazines | Ametryne |
| | | at photosystem II | | Atrazine |
| | | | | Cyanazine |
| | | | | Desmetryne |
| | | | | Dimethametryne |
| | | | | Prometon |
| | | | | Prometryne |
| | | | | Propazine |
| | | | | Simazine |
| | | | | Simetryne |
| | | | | Terbumeton |
| | | | | Terbuthylazine |
| | | | | Terbutryne |
| | | | | Trietazine |
| | | | Triazinones | Hexazinone |
| | | | | Metamitron |
| | | | | Metribuzin |
| | | | Triazolinone | Amicarbazone |
| | | | Uracils | Bromacil |
| | | | | Lenacil |
| | | | | Terbacil |
| | | | Pyridazinones | Pyrazon = chloridazon |
| | | | Phenyl-carbamates | Desmedipham |
| | | | | Phenmedipham |
| C2 | 7 | Inhibition of photosynthesis | Ureas | Chlorobromuron |
| | | at photosystem II | | Chlorotoluron |
| | | | | Chloroxuron |
| | | | | Dimefuron |
| | | | | Diuron |
| | | | | Ethidimuron |
| | | | | Fenuron |
| | | | | Fluometuron (see f3) |
| | | | | Isoproturon |
| | | | | Isouron |
| | | | | Linuron |
| | | | | Methabenzthiazuron |
| | | | | Metobromuron |
| | | | | Metoxuron |
| | | | | Monolinuron |
| | | | | Neburon |
| | | | | Siduron |
| | | | | Tebuthiuron |
| | | | Amides | Propanil |
| | | | | Pentanochlor |
| C3 | 6 | Inhibition of photosynthesis | Nitriles | Bromofenoxim (also M) |
| | | at photosystem II | | Bromoxynil |
| | | | | (also group M) |
| | | | | Ioxynil (also group M) |
| | | | Benzothiadiazinone | Bentazon |
| | | | Phenyl-pyridazines | Pyridate |
| | | | | Pyridafol |
| D | 22 | Photosystem-L-electron | Bipyridyliums | Diquat |
| | | diversion | | Paraquat |
| E | 14 | Inhibition of | Diphenylethers | Acifluorfen-na |
| | | protoporphyrinogen oxidase | | Bifenox |
| | | (PPO) | | Chlomethoxyfen |
| | | | | Fluoroglycofen-ethyl |
| | | | | Fomesafen |
| | | | | Halosafen |
| | | | | Lactofen |
| | | | | Oxyfluorfen |
| | | | Phenylpyrazoles | Fluazolate |
| | | | | Pyraflufen-ethyl |
| | | | N-phenylphthalimides | Cinidon-ethyl |
| | | | | Flumioxazin |
| | | | | Flumiclorac-pentyl |
| | | | Thiadiazoles | Fluthiacet-methyl |
| | | | | Thidiazimin |
| | | | Oxadiazoles | Oxadiazon |
| | | | | Oxadiargyl |
| | | | Triazolinones | Azafenidin |
| | | | | Carfentrazone-ethyl |
| | | | | Sulfentrazone |
| | | | Oxazolidinediones | Pentoxazone |
| | | | Pyrimidindiones | Benzfendizone |
| | | | | Butafenacil |
| | | | Others | Pyrazogyl |
| | | | | Profluazol |
| F1 | 12 | Bleaching: | Pyridazinones | Norflurazon |
| | | Inhibition of carotenoid | | |
| | | biosynthesis at the phytoene | | |
| | | desaturase step (PDS) | | |
| | | | Pyridinecarboxamides | Diflufenican |
| | | | | Picolinafen |
| | | | Others | Beflubutamid |
| | | | | Fluridone |
| | | | | Flurochloridone |
| | | | | Flurtamone |
| F2 | 28 | Bleaching: | Triketones | Mesotrione |
| | | Inhibition of 4- | | Sulcotrione |
| | | hydroxyphenyl-pyruvate- | | |
| | | dioxygenase (4-HPPD) | | |
| | | | Isoxazoles | Isoxachlortole |
| | | | | Isoxaflutole |
| | | | Pyrazoles | Benzofenap |
| | | | | Pyrazolynate |
| | | | | Pyrazoxyfen |
| | | | Others | Benzobicyclon |
| F3 | 11 | Bleaching: | Triazoles | Amitrole |
| | | Inhibition of carotenoid | | (in vivo inhibition of |
| | | biosynthesis (unknown | | Lycopene cyclase) |
| | | target) | | |
| | 13 | | Isoxazolidinones | Clomazone |
| | | | Ureas | Fluometuron (see C2) |
| | | | Diphenylether | Aclonifen |
| G | 9 | Inhibition of EPSP synthase | Glycines | Glyphosate |
| | | | | Sulfosate |
| H | 10 | Inhibition of glutamine | Phosphinic acids | Glufosinate-ammonium |
| | | synthetase | | Bialaphos = bilanaphos |
| I | 18 | Inhibition of DHP | Carbamates | Asulam |
| | | (dihydropteroate) synthase | | |
| K1 | 3 | Microtubule assembly | Dinitroanilines | Benefin = benfluralin |
| | | inhibition | | Butralin |
| | | | | Dinitramine |
| | | | | Ethalfluralin |
| | | | | Oryzalin |
| | | | | Pendimethalin |
| | | | | Trifluralin |
| | | | Phosphoroamidates | Amiprophos-methyl |
| | | | | Butamiphos |
| | | | Pyridines | Dithiopyr |
| | | | | Thiazopyr |
| | | | Benzamides | Propyzamide = pronamide |
| | | | | Tebutam |
| | 3 | | Benzenedicarboxylic acids | DCPA = chlorthal- |
| | | | | dimethyl |
| K2 | 23 | Inhibition of mitosis/ | Carbamates | Chlorpropham |
| | | microtubule organisation | | Propham |
| | | | | Carbetamide |
| K3 | 15 | Inhibition of cell division | Chloroacetamides | Acetochlor |
| | | (Inhibition of VLCFAs; see | | Alachlor |
| | | Remarks) | | Butachlor |
| | | | | Dimethachlor |
| | | | | Dimethanamid |
| | | | | Metazachlor |
| | | | | Metolachlor |
| | | | | Pethoxamid |
| | | | | Pretilachlor |
| | | | | Propachlor |
| | | | | Propisochlor |
| | | | | Thenylchlor |
| | | | Acetamides | Diphenamid |
| | | | | Napropamide |
| | | | | Naproanilide |
| | | | Oxyacetamides | Flufenacet |
| | | | | Mefenacet |
| | | | Tetrazolinones | Fentrazamide |
| | | | Others | Anilofos |
| | | | | Cafenstrole |
| | | | | Indanofan |
| | | | | Piperophos |
| L | 20 | Inhibition of cell wall | Nitrites | Dichlobenil |
| | | (cellulose) synthesis | | Chlorthiamid |
| | 21 | | Benzamides | Isoxaben |
| | | | Triazolocarboxamides | Flupoxam |
| M | 24 | Uncoupling (Membrane | Dinitrophenols | Dnoc |
| | | disruption) | | Dinoseb |
| | | | | Dinoterb |
| N | 8 | Inhibition of lipid synthesis- | Thiocarbamates | Butylate |
| | | not ACCase inhibition | | Cycloate |
| | | | | Dimepiperate |
| | | | | EPTC |
| | | | | Esprocarb |
| | | | | Molinate |
| | | | | Orbencarb |
| | | | | Pebulate |
| | | | | Prosulfocarb |
| | | | | Thiobencarb = |
| | | | | benthiocarb |
| | | | | Tiocarbazil |
| | | | | Triallate |
| | | | | Vernolate |
| | | | Phosphorodithioates | Bensulide |
| | | | Benzofuranes | Benfuresate |
| | | | | Ethofumesate |
| | 26 | | Chloro-Carbonic-acids | Tca |
| | | | | Dalapon |
| | | | | Flupropanate |
| O | 4 | Action like indole acetic | Phenoxy-carboxylic-acids | Clomeprop |
| | | acid (synthetic auxins) | | 2,4-D |
| | | | | 2,4-DB |
| | | | | Dichlorprop = 2,4-DP |
| | | | | MCPA |
| | | | | MCPB |
| | | | | Mecoprop = MCPP = |
| | | | | CMPP |
| | | | Benzoic acids | Chloramben |
| | | | | Dicamba |
| | | | | TBA |
| | | | Pyridine | Clopyralid |
| | | | carboxylic acids | Fluroxypyr |
| | | | | Picloram |
| | | | | Triclopyr |
| | | | Quinoline carboxylic acids | Quinclorac |
| | | | | (also group L) |
| | | | | Quinmerac |
| | | | Others | Benazolin-ethyl |
| P | 19 | Inhibition of auxin transport | Phthalamates | Naptalam |
| | | | Semicarbazones | Diflufenzopyr-Na |
| R | | . . . | . . . | . . . |
| S | | . . . | . . . | . . . |
| . | | . . . | . . . | . . . |
| Z | 25 | Unknown | Arylaminopropionic acids | Flamprop-M-methyl/- |
| | | | | isopropyl |
| | 8 | | Pyrazolium | Difenzoquat |
| | 17 | | Organoarsenicals | Dsma |
| | | | | Msma |
| | 27 | | Others | Bromobutide |
| | | | | (chloro)-flurenol |
| | | | | Cinmethylin |
| | | | | Cumyluron |
| | | | | Dazomet |
| | | | | Dymron = daimuron |
| | | | | Methyl-dimuron = |
| | | | | Methyl-dymron |
| | | | | Etobenzanid |
| | | | | Fosamine |
| | | | | Metam |
| | | | | Oxaziclomefone |
| | | | | Oleic acid |
| | | | | Pelargonic acid |
| | | | | Pyributicarb |

The following additional herbicides were classified in the February 2000 meeting of the HRAC and WSSA groups:

| HRAC (WSSA) Classification | Herbicide |
|---|---|
| A (1): | Tepraloxidim |
| B (2): | Foramsulfuron |
| | Tritosulfuron |
| | Pyriftalid |
| C1 (5): | Amicarbazone |
| E (14): | Benzfendizone |
| | Butafenacil |
| | Pyrazogyl |
| | Profluazol |
| F1 (12): | Picolinafen (AC900001, BAS 700) |
| F1: | Pyridinecarboxamides instead of |
| | nicotinanilides |
| F1: | Triazolinones instead of triazolopyridines |
| K3 (15): | Indanofan |
| Inhibition of the synthesis of | Chloroacetamide |
| very-long-chain fatty acids | |
| (VLCFAs).ⁱ | |

### APPENDIX II

### Practical Use of SNNS Software

Procedure to Process NMR Files

First, the phase and baseline of each frequency domain NMR spectrum are manually corrected. Then, the processed spectra are exported by the spectrometer software in the JCAMP file format and automatically processed using a package of Perl scripts that prepare the data for presentation to the Stuttgart Neural Network Simulator software, as follows:

1. Run Multicom najdx—delivers vector to subdirectory /nn/jdc.

2. Run rename.csh to change the filename from 1 to 2 digit file numbering.

3. Make vector: runjdc2vect.gmo [-o todir]filename. Will produce a set of 3 files from each spectrum with the file extensions: *.asc, *.asg, *.outnode.

Procedure for NN Analysis

1) Definition of a NN topology: three layers, comprising one input layer with 1080 nodes, one hidden layer with six or twelve nodes, one output layer with one node for each class (six classes in the examples presented here). The NN units were represented by a logistic activation function, and all units were fully connected with the adjacent layer. The input layer represents the spectral information and is initialized with the pattern created as described above. For training the NN, the output layer is initialized with a corresponding vector that describes the desired answer of the NN for a given input vector. For example, the definition of the output nodes may be as follows: 1^{st} node: Untreated: 2^{nd} node: AHAS inhibitor, 3^{rd} node: ACCase inhibitor, 4^{th} node: EPSPS inhibitor, 5^{th} node: PSII inhibitor, 6^{th} node: Dead Plant. Note that the enzyme abbreviations are defined in the legend to Table I. The hidden layer and all connections are initialized using random values in the range of [−1, 1].

2) Presentation of a training set (a subset of the pattern, with known assignments for the output nodes) to this NN, and the training, i.e. initialization and adjustment of the weights of the connections in an iterative manner using a learning function until convergence or a step limit is reached. During this step, a validation set (a subset of the patterns different from those used as the training set) can, optionally, be periodically presented to the NN to gauge the performance of the NN and detect possible “overtraining”.

3) A test set (a pattern for which the output nodes are not defined, i.e. the mode-of-action unknown) can then be presented to the NN for classification.

Use the “Resilient Backpropagation” (Rprop) learning function for training the NN with the following learning parameters:

Initial update value Δ₀=0.1.

Limit for the maximum step size, Δₘₐₓ=50.

Weight decay exponent α=4 (a value in the range of 3-9 can be tried).

The training is done in cycles of 25 steps, after which the network is saved. The validation set is presented and the network error on the validation set is calculated. This procedure is repeated for up to 20 epochs (500 cycles total) and the network that produced the minimum error on the validation set is kept.

1. Run mkpat filename or *.asc>filename.pat

2. options -n [# of points to average] -p [, #, #, #] (for start, end (water), start, end)

3. e.g. mkpat -n5 -p 965 3440, 4330, 7254 filename>newname.pat.

4. Edit the file list to make 2 sets of patterns: test and train 1s -1 na0608*.asc>na0608files.lis.

5. Prepare patterns 1 and 2 with Comm -23 *files* *fil2* >*fil1*

Procedure to Run SNNS

1. Running SNNS Interactively

Log on to the SGI workstation “max”, change to the neutral network working directory /nm01/data/araniban/nn/nnruns/run# when run# is the current run number (e.g. run7). Type snns from the operating directory, left click on the banner window to remove it.

Under thefile pull-down menu:

Load a network file *.net via the net button (e.g. net23.net for 23 output nodes).

Load one or two pattern files *.pat with load button, and use one for training and the other for validation. For testing, load a different pattern file in order to compare efficiency of training.

Load a network configuration file (*.cfg) via the cfg button.

Begin the network training by clicking the all button.

Running SNNS in Batch Mode

The Perl script RUNME was written to automate the running of SNNS via the batchman utility. RUNME also generates useful output file formats. It is called by typing “RUNME run#” (e.g. RUNME run7) and assumes that the files SNNS_config.cfg, moa.pat, run#.names, net23.net, run#.bat, and t1.bat are present in the same directory.

The above examples are intended to illustrative of the invention and are not intended to limit the scope of the appended claims.

Patents:
Plant, Food and Agriculture Related Metabolite Profiling

| | | |
|---|---|---|
| WO2000001302 | Reynnells et al | 2000 |
| GB2335491 | Syms | 1999 |
| US5900634 | Solaman et al | 1999 |
| JP11271298 | Takeda et al | 1999 |
| JP09218192 | Takahashi et al | 1997 |
| JP95158138 | Horigane et al | 1995 |
| WO9531710 | Sjoeberg et al | 1995 |
| US5252490 | Brenneison et al | 1993 |
| WO9202886 | Meyer et al | 1992 |
| US5025214 | Conner et al | 1991 |
| WO8403563 | Colby et al | 1984 |
| US4314027 | Stahr | 1982 |

Metabolic Profiling in Humans

| | | |
|---|---|---|
| US5887588 | Ala-Korpela et al | 1999 |
| WO9950437 | Kristal et al | 1999 |
| US5687716 | Beving et al | 1997 |
| US5456252 | Grundfest et al | 1995 |

### JOURNAL ARTICLES

Metabolite Profiling in Plants

Lozano, J; Novic, M; Ruis, FX; Zupan, J. Modeling Metabolic Energy by Neural Networks. Chemom. Intell.Lab. Syst., 1995, 28, 1, p.61-72.

Sauter, H; Lauer, M; Fritsch, H. Metabolic Profiling in Plants. ACS Symposium Series 443, Baker, Feynes, Moberg Eds.

Hole, S. J. W.; Howe, W. A.; Stanley, P.D.; Hadfield, S. T. J. Biomol. Screening, 2000, 5, p.335-342.

Metabolic Profiling in Humans

Ala-Korpela, M; Chaugani, KK; Hiltunan, Y; Bell, J D; Fuller, B J; Bryant, D J. Assessment of Quantitative Artificial Neural Network Analysis in a Metabolically dynamic ex vivo 31P NMR pig liver study. Magn. Reson. Med(US), 1997, 38, 5, p. 840-845.

Anthony, M L; Rose V S; Nicholson, J K; Lindon, J C. Classification of Toxin-induced changes in H-1 NMR Spectra of Urine using an Artificial Neural Network. J. Pharmaceutical and Biomedical Analysis, 1995, 13, N3, p.205-211.

Austin, A J; Piergentili, D; Ward, A C; Kara, B; Glassey, J. Monitoring and Control of Stress in Recombinant E. coli during Fermentation using pyrolysis mass spectrometry and Artificial Neural Networks. IChemE (Symposium), 1998, p.215-224 IChemE Publ.

Bakken, A; Axelson, D; Kristad, K A; Brodtkorb,E; Muller, B; Asaly, J; Gribbestad, I S. Application of Neural Network Analysis to in-vitro H-1 Magnetic Resonance Spectroscopy of Epilepsy patients. Epilepsy Res., 1999, 35, 3, 245-252.

Bales J R, Higham M, Howe I, Nicholson J K, Sadler P J. (1984) Clin. Chem., 30, 426-32.

Bamforth F J; Dorian V; Valiance H; Wiahort D S. Diagnosis of Inborn Errors of Metabolism using 1H NMR Spectroscopic Analysis of Urine. J. Inherited Metab. Dis. 1999, 22, 3, 297-301.

Dhar S; Nygren P; Csoka K; Botling J; Nilsson K; Larsson R. Anti-cancer Drug Characterization Using a Human Cell-line panel representing defined types of Drug Resistance. British J. Cancer, 1996, 74, 6, 888-896.

El-Deredy W; Ashmore S M; Branston N M; Darling J L; Williams S R; Thonas D G. Pretreatment prediction of the chemotherapeutic response of human glioma cell cultures using Nuclear Magnetic Resonance spectroscopy and Artificial Neural Networks. Cancer Res.,(US), 1997, 10, 5, p. 99-124.

El-Deredy W. Pattern Recognition Approaches in Biomedical and Clinical Magnetic Resonance Spectroscopy: A Review. NMR Biomed. (Eng.)., 1997, 10, 5, p. 99-124.

Fiehn, O., Kopka, J., Doermann, P., Altmann, T., Trethewey, R. N., Willmitzer, L. (2000) Nature Biotechnology 18, 1157-1161.

Fu, D C; Barford, J P. A Hybrid Neural Network—A First Principles Approach for Modeling of Cell Metabolism. Comput. Chem. Eng., 1996, 20, 6/7, p. 951-8.

Geers, R; Decanniere, C; Rosier, A; Ville, H; Van Hecke, P; Vandesande, F; Jourquin, J. Variability of Energy Metabolism AND Nuclear T3-receptors within the Skeletal Muscle Tissue of Pigs different with respect to the halothane Gene. J Anim Sci., 1996, 74, 4, 717-720

Geers, R; Decanniere, C; Truyen, B; Ville, H; Van Hecke, P; Jourquin. In vivo measurement of energy metabolism of Skeletal Muscle Tissue during malignant hyperthermia of Pigs. EAAP Publ., 1994, 76(Energy Metabolism of Farm Animals), 23-26.

Gobburu J V; Chen E P; Emile P. Artificial Neural Networks as a Novel Approach to Integrated Pharmacokinetic-Pharmacodyamic Analyses. J. Pharm. Sci., 1996, 85, 5, p. 505-10.

Gribbestad I S; Sitter B; Lundgren S; Krane J; Axelson D. Metabolite Composition in Breast Tumors examined by Proton Nuclear Magnetic Resonance Spectroscopy. Anticancer Res.(Greece), 1999, 19, 3A, p.1737-46.

Hagimori S; Fukuda T; Kuroda C; Ishida M. State Recognition by Neural Networks for byproduct formation in fed-batch Yeast Fermentation. Kagaku Kogahu Ronbunshu, 1993, 19, 3, p. 353-9.

Hertz, J; Heller, J; Kjoer, T; Richmond, B. Information Spectroscopy of Single Neurons. Int'l Journal of Neural Systems, 1995, 6, Supp. P, 123-132.

Huang, S. Methodology for Developing Kinetic Models for Microbial Reaction Systems. Guoli Taiwan Daxue Gongcheng Xuekan, 1996, 68, 65-90

Haug H, Schramm C. (1975) Clin. Chem., 21, 1025.

Holmes, E., Foxall, P. J. D., Neild, G. H., Beddell, C., Sweatman, B. C., Rahr, E., Lindon, J. C., Spraul, M., and Nicholson, J. K. (1994) Analytical Biochemistry, 220, 284-296.

Kaartinen, J; Miserisova, S; Oja, J M W; Usenius, J P; Kauppinen, R A, Hiltunen, Y. Automated Quantification of Human Brain Metabolites by Artificial Neural Network Analysis from in vivo Single-voxel H-1 NMR spectra. Journal of Magnetic Resonance, 1998, 134, 1, 176-179.

Kang, S G; Kenyon, R G W; Ward, A C; Lee, K J. Analysis of Differentiation State in Streptomyces albidoflavus SMF301 by the combination of Pyrolysis Mass Spectrometry and Neural Networks. J. Biotechnology, 1998, 62, 1, 1-10.

Kari, S; Olsen, N J; Park, J H. Evaluation of Muscle Diseases using Artificial Neural Network Analysis of P-31 MR Spectroscopy Data. Magnetic Resonance in Medicine, 1995, 34, 5, 664-672.

Kell, D B; Davey, C L; Goodacre, R; Sauro, H M. When Going Backwards MEANS Progress: On the solution of Biochemical Inverse Problems using Artificial Neural Networks. Modern Trends in Biothernkinetics, 1993, 109-114.

Kurtanjek Z. Modeling and Control by Artificial Neural Networks in Biotechnology. Comput. Chem Eng., 1993, 18, S627-S631.

Lee, H-S., Chung, Y. H., Kim C. Y., (1991) Hepatology, 14, 68.

Li, H; Godfrey, T G; Godfrey, D A; Rubin A M. Quantitative Changes of Amino acid distribution in the RAT Vestibular Nuclear Complex after Unilateral Vestibular Ganglionnectomy. J. Neuorchemistry. 1996, 66,4, 1550-1564

Lisboa P J; Branston, N; El-Deredy W; Vellido, A; Characterization with NMR Spectroscopy: Current State and Future Prospects for the Application of Neural Networks Analysis. IEEE International Conference on Neural NETWORKS, 1997, 3, 1385-1390.

Lisboa P; Kirby S P; Vellido, A; Lee, Y Y; El-Deredy W. Assessment of Statistical and Neural Networks methods in NMR spectral classification and Metabolite Selection. NMR Biomed., 1998, 11, 4-5, 225-234.

Mansfield, J. R., Sowa, M. G., Scarth, G. B., Somoijai, R. L., Mantsch, H. H. (1997) Analytical Chemistry 69, 3370-3374.

Maquelin, K; Choo-Smith L P, van Vreeswijk, T; Endtz H P; Smith, B; Bennett, R; Bruining, H A; Puppels G J. Raman Spectroscopic Method for Identification of Clinically relevant microorganisms growing on Solid Culture Medium. Analytical Chemistry, 2000, 72, 1, 12-19.

McGovern, A C; Ernill, R; Kara, B V; Kell, D B; Goodacre, R. Rapid Analysis of the Expression of Heterologous Proteins in E. coli using Pyrolysis Mass Spectrometry and Fourier transform Infrared Spectroscopy with Chemometrics: application to alpha 2-interferon production. J. Biotechnol., 1999, 72, 3, 157-167

Mendes, P; Kell, D B. On the Analysis of the Inverse problem of Metabolic Pathways using Artificial Neural Networks. Biosystems, 1996, 38, 1, 15-28.

Munk, M E; Madison, M S; Robb, E W. The Neural Network as a Tool for Multispectral Interpretation. J. Chem. Inf. Comput. Sci., 1996, 36, 2, 231-238.

Nicholson, J. K., Wilson I. D. (1989) Prog. NMR Spectr. 21, 449-501.

Nicholson, J. et al. (1995) J. Pharm. & Biomed. Anal., 13, 205-211.

Ohsaka, A., Yoshikawa K., Matsuhashi T., (1979) Jpn. J. Med. Sci. Biol., 32, 305-309.

Pierard, C; Champagnat, J; Denavit-Saubie, M; Gillet, B; Beloeil, J C; Guezennec, C Y; Barrere, B; Peres, M. Brain stem Energy Metabolism response to Acute Hypoxia in Anaesthetized Rats: a 31-P NMR Study. Neuroreport, 1995, 7, 1, 281-285.

Rabenstein, D. L., Millis, K. K., Strauss, E. J. (1988) Anal Chem., 60, 1380A-1391A.

Riedmiller, M., Proceedings of the SNNS 1993 workshop, Riedmiller, & Braun, Proceedings of the IEEE International Conference on Neuronal Networks 1993]

Sackett, R E; Rogers, S K; Desimio, M S; Raymer, J H; Ruck, D W; Kabrisky, M; Bleckmann, C A. Neural Network Analysis of Chemical Compounds in Nonbreathing Fisher-344 rat breach. SPIE Proceedings Series, 1996, 2760, 386-397.

Savchenko, A A; Shakina, N A; Rossien, D A. Neural Network Classification of Patients with Chronic Non-specific lung diseases using Immunological Parameters of Blood and Activities of Lymphocyte Metabolic Enzymes. Vopr. Med. Khim., 1998, 44, 3, 267-273.

Shaw, R. A., Kotowich, S., Eysel, H. H, Jackson, M., Thomson, G. T. D. (1995) Rheumatol. Int. 15, 159-165.

Somorjai, R. L., Dolenko, B., Nikulin, A. K., Pizzi, N., Scarth, G., Zhilkin, P., Halliday, W., Fewer, D., Hill, N., Ross, I., West, M., Smith, I. C. P., Donnelly, S. M., Kuesel, A. C., Brière, K. M. (1996) JMRI 6, 437-444.

Syu, Mei-J; Hou, C. a Neural Network Study on the Dynamic Identification of a Fermentation System. Bioprocess Eng., 1997, 17, 4, 203-213.

Thompson, M L; Kramer, M A. Modeling Chemical Process Using Prior knowledge and Neural Networks. AIChE j., 1994, 40, 8, 1328-1340.

Timmins, E M; Howell, S A; Alsberg, B K; Noble, W C; Goodacre, R. Rapid Differentiation of Closely related Candida species and strains by Pyrolysis Mass Spectrometry and Fourier transform-infrared Spectroscopy. J. Clinical Microbiology, 1998, 36, 2, 367-374.

Torri, G M; Torri, J; Gulian, J M; Vion-Dury, 3; Viout, P; Cozzone, P J. Magnetic Resonance Spectroscopy of Serum and Acute-phase Proteins revisited: a multiparametric statistical analysis of Metabolite variations in inflammatory, infectious and miscellaneous diseases. Clinica Chimica Acta, 1999, 279, 1-2, 77-96

Usenius, J; Tuchimetsa, S; Vainio, P; Ala-Korpela, M; Hiltunen, Y; Kauppinen, R A. Automated Classification of Human Brain Tumors by Neural Network Analysis using in vivo 1H Magnetic Resonance Spectroscopic Metabolite Phenotypes. NeuroReport, 1996, 7, 10, 1597-1600.

Woodward, A M; Gilbert, R J; Kell, D B; Genetic Programming as an Analytical Tool for Non-Linear Dielectric Spectroscopy. Bioelectrochem. Bioenerg., 1999, 48, 2, 389-396.

Woodward, A M; Jones, A; Zhang, X Z; Rowland, J; Kell, D. B. Rapid and Noninvasive Quantification of Metabolic Substrates in Biological Cell Suspensions using non-linear dielectric Spectroscopy with Multivariate Calibration and Artificial Neural Networks. Bioelectrochemistry and Bioenerg., 1996, 40, 2, 99-132.

Zupan, J; Gasteiger, J. Neural Networks: A new method for solving chemical problems or just a passing phase? Analytica chimica acta, 1991, 248, 1, 1-30.

### Neural Network Analysis and Analytical Techniques in Plants/Agriculture

Alnasser, Ghassan Hamki. Use of Thermal Desorption/Gas Chromatography/Mass Spectrometry, Honey Bees, and Artificial Neural Networks(ANN) in Assessing EcoSystem Contamination. Dissertation Abstracts International, Volume 59/11-B, p5823.

Zell, Andreas, Simulation Neuronaler Netze.R Oldenbourg Verlag Muenchen, Germany, 2000 (German)

Rumelhart, D. E., McClleland, J. J L., Parallel Distributed Processing, 1, MIT Press, 1968 (English)

Braun, H., Reidmiller, M., Rprop: A Fast adaptive learning Algorithm. In Proc. of the International Symposium on Computer and Information Service V11, 1992.

Braun, H., Reidmiller, M., Rprop: A Fast and Robust Backpropogation Learning Strategy In Proc. of the ACNN, 1993

Stuttgart Neural Network Simulator (SNNS), User Manual, Version 4.1, University of Stuttgart.

We claim:

1. A metabolic profiling method for identifying a metabolic state of a subject biological sample, wherein said method comprises analyzing in an automated pattern recognition system data obtained from the subject biological sample by a spectroscopic or chromatographic technique in comparison to data obtained from a plurality of other known biological samples by the spectroscopic or chromatographic technique to determine a comparable metabolic state, wherein the biological samples are obtained from organisms grown under controlled conditions, and wherein the data is a compilation of a plurality of observed metabolites.

2. The method of claim 1, wherein the chromatographic technique is gas chromatography.

3. The method of claim 1, wherein the spectroscopic technique is nuclear magnetic resonance spectroscopy.

4. The method of claim 1, wherein the spectroscopic technique is mass spectroscopy.

5. The method of claim 1, wherein said method employs data obtained from both chromatographic and spectroscopic techniques.

6. The method of claim 1, wherein the pattern recognition analysis system comprises a neural network analysis.

7. The method of claim 1, wherein the metabolic state is selected from the group consisting of:
a. inhibition of acetyl CoA carboxylase (ACCase);
b. inhibition of acetolactate synthase (ALS) or acetohydroxyacid synthase (AHAS);
C. inhibition of photosynthesis at photosystem II;
d. photosystem-I-electron diversion;
e. inhibition of protoporphyrinogen oxidase (PPO);
f. inhibition of carotenoid biosynthesis at the phytoene desaturase step (PDS);
g. inhibition of 4-hydroxyphenyl-pyruvate-dioxygenase (4-HPPD);
h. inhibition of carotenoid biosynthesis;
i. inhibition of EPSP synthase;
j. inhibition of glutamine synthetase;
k. inhibition of DHP (dihydropteroate) synthase;
l. microtubule assembly inhibition;
m. inhibition of mitosis/microtubule organization;
n. inhibition of cell division;
o. inhibition of VLCFAs;
p. inhibition of cell wall (cellulose) synthesis;
q. uncoupling (membrane disruption);
r. inhibition of lipid synthesis—not ACCase inhibition;
s. action like indole acetic acid (synthetic auxins); and
t. inhibition of auxin transport;

8. The method of claim 1 wherein previously unknown metabolic states are identified as distinguished from known metabolic states associated with herbicide modes-of-action in an artificial neural network simulation.

9. The method of claim 1, wherein the biological samples are obtained from organisms of the same species.

10. The method of claim 1, wherein the sample is from a fungi tissue.

11. The method of claim 1, wherein the sample is from a yeast tissue.

12. The method of claim 1, wherein the sample is from a bacteria.

13. The method of claim 1, wherein the sample is from an animal tissue.

14. The method of claim 1, wherein the sample is from a plant tissue.

15. The method of claim 14, wherein said plant tissue is plant protoplast.

16. The method of claim 14, wherein said plant tissue is whole plant.

17. The method of claim 14, wherein said plant tissue is a partial plant.

18. The method of claim 14, wherein said plant tissue is callus tissue.

19. The method of claim 14, wherein said plant tissue is a cell suspension culture.

20. A method for determining the metabolic mode of action of a compound wherein said method comprises the method of claim 1 and said subject biological sample is from an organism treated with the compound, and said subject metabolic state indicates the metabolic mode of action of the compound.

21. A method for the determining the metabolic stress response in plants to stimuli wherein said method comprises the method of claim 1 and said subject biological sample is from an organism exposed to the stimuli, and said subject metabolic state indicates the metabolic stress response to the stimuli.

22. The method of claim 21, wherein the stimuli is a change in temperature, salinity or moisture.

23. A metabolic profiling process wherein said process comprises
a. growing organisms under controlled conditions;
b. treating a control subset of the organisms with known bioregulators;
c. treating a subject subset of the organisms with an uncharacterized bioregulator;
d. preparing samples of tissues of the subsets of the organisms;
e. obtaining spectroscopic or chromatographic data of a plurality of metabolites from the samples;
f. training an automated pattern recognition system by association of the spectroscopic or chromatographic data from the control subset of the organisms treated with the known bioregulator to determine a control metabolic profile;
g. generating a mathematical model from the trained pattern recognition system based on spectroscopic or chromatographic data of the control subset of the organisms associated with the control metabolic profile;
h. applying the mathematical model to the spectroscopic or chromatographic data of the subject subset of the organisms to determine the subject metabolic profile; and,
i. comparing the subject metabolic profile to the control metabolic profile to determine the metabolic association of the uncharacterized bioregulator to the known bioregulator.

24. The method of claim 23, wherein the chromatographic technique is gas chromatography.

25. The method of claim 23, wherein the spectroscopic technique is nuclear magnetic resonance spectroscopy.

26. The method of claim 23, wherein the spectroscopic technique is mass spectroscopy.

27. The method of claim 23, wherein said method employs data obtained from both chromatographic and spectroscopic techniques.

28. The method of claim 23, wherein the pattern recognition analysis system comprises a neural network analysis.

29. The method of claim 23, wherein the metabolic profile results from a metabolic state selected from the group consisting of:
a. inhibition of acetyl CoA carboxylase (ACCase);
b. inhibition of acetolactate synthase (ALS) or acetohydroxyacid synthase (AHAS);
c. inhibition of photosynthesis at photosystem II;
d. photosystem-I-electron diversion;
e. inhibition of protoporphyrinogen oxidase (PPO);
f. inhibition of carotenoid biosynthesis at the phytoene desaturase step (PDS);
g. inhibition of 4-hydroxyphenyl-pyruvate-dioxygenase (4-HPPD);
h. inhibition of carotenoid biosynthesis;
i. inhibition of EPSP synthase;
j. inhibition of glutamine synthetase;
k. inhibition of DHP (dihydropteroate) synthase;
l. microtubule assembly inhibition;
m. inhibition of mitosis/microtubule organization;
n. inhibition of cell division;
o. inhibition of VLCFAs;
p. inhibition of cell wall (cellulose) synthesis;
q. uncoupling (membrane disruption);
r. inhibition of lipid synthesis—not ACCase inhibition;
s. action like indole acetic acid (synthetic auxins); and
t. inhibition of auxin transport.

30. The method of claim 23, wherein previously unknown metabolic profiles are identified as distinguished from known metabolic profiles associated with herbicide modes-of-action in an artificial neural network simulation.

31. The method of claim 23, wherein the biological samples are obtained from organisms of the same species.

32. The method of claim 23, wherein the sample is from a fungi tissue.

33. The method of claim 23, wherein the sample is from a yeast tissue.

34. The method of claim 23, wherein the sample is from a bacteria.

35. The method of claim 23, wherein the sample is from an animal tissue.

36. The method of claim 23, wherein the sample is from a plant tissue.

37. The method of claim 36, wherein said plant tissue is plant protoplast.

38. The method of claim 36, wherein said plant tissue is whole plant.

39. The method of claim 36, wherein said plant tissue is a partial plant.

40. The method of claim 36, wherein said plant tissue is callus tissue.

41. The method of claim 36, wherein said plant tissue is a cell suspension culture.

42. A metabolic profiling process wherein said process comprises
a. growing organisms under controlled conditions;
b. selecting a control subset of the organisms with known phenotypic or genotypic traits;
c. selecting a subject subset of the organisms with a potential unknown genetic modification or altered phenotype;
d. preparing samples of tissues of the subsets of the organisms;
e. obtaining spectroscopic or chromatographic data of a plurality of metabolites from the samples;
f. training an automated pattern recognition system by association of the spectroscopic or chromatographic data from the control subset of the organisms to determine a control metabolic profile;
g. generating a mathematical model from the trained pattern recognition system based on spectroscopic or chromatographic data of the control subset of the organisms associated with the control metabolic profile;
h. applying the mathematical model to the spectroscopic or chromatographic data of the subject subset of the organisms to determine the subject metabolic profile; and,
i. comparing the subject metabolic profile to the control metabolic profile to determine the metabolic association of the potential unknown genetic modification or altered phenotype to the known phenotypic or genotypic traits.

43. The method of claim 42, wherein the chromatographic technique is gas chromatography.

44. The method of claim 42, wherein the spectroscopic technique is nuclear magnetic resonance spectroscopy.

45. The method of claim 42, wherein the spectroscopic technique is mass spectroscopy.

46. The method of claim 42, wherein said method employs data obtained from both chromatographic and spectroscopic techniques.

47. The method of claim 42, wherein the pattern recognition analysis system comprises a neural network analysis.

48. The method of claim 42, wherein the metabolic profile results from a metabolic state selected from the group consisting of:
a. inhibition of acetyl CoA carboxylase (ACCase);
b. inhibition of acetolactate synthase (ALS) or acetohydroxyacid synthase (AHAS);
c. inhibition of photosynthesis at photosystem II;
d. photosystem-I-electron diversion;
e. inhibition of protoporphyrinogen oxidase (PPO);
f. inhibition of carotenoid biosynthesis at the phytoene desaturase step (PDS);
g. inhibition of 4-hydroxyphenyl-pyruvate-dioxygenase (4-HPPD);
h. inhibition of carotenoid biosynthesis;
i. inhibition of EPSP synthase;
j. inhibition of glutamine synthetase;
k. inhibition of DHP (dihydropteroate) synthase;
l. microtubule assembly inhibition;
m. inhibition of mitosis/microtubule organization;
n. inhibition of cell division;
o. inhibition of VLCFAs;
p. inhibition of cell wall (cellulose) synthesis;
q. uncoupling (membrane disruption);
r. inhibition of lipid synthesis—not ACCase inhibition;
s. action like indole acetic acid (synthetic auxins); and
t. inhibition of auxin transport.

49. The method of claim 42, wherein previously unknown metabolic states are identified as distinguished from known metabolic states associated with herbicide modes-of-action in an artificial neural network simulation.

50. The method of claim 42, wherein the biological samples are obtained from organisms of the same species.

51. The method of claim 42, wherein the sample is from a fungi tissue.

52. The method of claim 42, wherein the sample is from a yeast tissue.

53. The method of claim 42, wherein the sample is from a bacteria.

54. The method of claim 42, wherein the sample is from an animal tissue.

55. The method of claim 42, wherein the sample is from a plant tissue.

56. The method of claim 55, wherein said plant tissue is plant protoplast.

57. The method of claim 55, wherein said plant tissue is whole plant.

58. The method of claim 55, wherein said plant tissue is a partial plant.

59. The method of claim 55, wherein said plant tissue is callus tissue.

60. The method of claim 55, wherein said plant tissue is a cell suspension culture.

61. A database of metabolic responses comprising data generated from the method of claim 1, claim 23 or claim 42.

62. The database of claim 61 wherein the genetic alteration comprises a gene mutation.

63. The database of claim 61 wherein the genetic alteration comprises a gene deletion.

64. The database of claim 61 wherein the genetic alteration comprises a gene insertion.

65. The database of claim 61 wherein the genetic alteration comprises gene activation change.

66. The database of claim 65 where the gene activation change comprises a change in transcription factors.

67. The database of claim 65 where the gene activation change comprises a change in promoters.

68. The database of claim 61 wherein the genetic alteration comprises a genetic modification.

69. The database of claim 68 wherein the genetic modification comprises knockout of gene activity.

70. The database of claim 68 wherein the genetic modification comprises inactivation of gene activity.

71. The database of claim 61 wherein the genetic alteration comprises insertion of genes.
